(19) United States
(12) Patent Application Publication
(10) Pub. No.: US 2026/0216643 A1
(43) Pub. Date: Jul. 30, 2026

(54) PROCESS FOR REMOVING CARBON DIOXIDE FROM A LIQUID AQUEOUS STREAM

(71) Applicant: BASF SE, Ludwigshafen am Rhein (DE)

(72) Inventors: Georg SIEDER, Ludwigshafen am Rhein (DE); Jonas SCHWABEN, Rhodt unter Rietburg (DE); Hermann LUYKEN, Ludwigshafen am Rhein (DE); Walter KOCH, Ludwigshafen am Rhein (DE)

(21) Appl. No.: 19/145,206

(22) PCT Filed: Jan. 3, 2024
(86) PCT No.: PCT/EP2024/050095

§ 371 Date: Jul. 2, 2025

(30) Foreign Application Priority Data
Jan. 4, 2023 (EP) 23150310.3

(51) Int. Cl. B01D 53/62; C02F 1/02; C02F 1/20; C07C 211/12

(52) U.S. Cl. CPC B01D 53/62; C02F 1/02; C02F 1/20; C07C 211/12; B01D 2257/504; C02F 2209/02

(57) ABSTRACT

Process for removing carbon dioxide from a liquid aqueous stream A process for removing carbon dioxide from a liquid aqueous stream comprising at least one hexane-1,6-diamine species which comprises at least one carbon dioxide derivative of hexane-1,6-diamine, the process comprising providing the liquid aqueous stream exhibiting a carbon dioxide loading c_{L1}, wherein 0.5≤c_{L1}≤2.5, wherein the liquid aqueous stream L₁ to be subjected to heating according to (ii) has a temperature T_{L1}<T according to (ii); and heating the liquid aqueous stream to a temperature T≥90° C. at a pressure p≥0.5 bar(abs), obtaining a vapor stream V comprising carbon dioxide, and obtaining a liquid aqueous stream L₂ exhibiting a carbon dioxide loading c_{L2}, wherein c_{L2}≤0.1.

The present invention relates to a process for removing carbon dioxide from a liquid aqueous stream comprising at least one hexane-1,6-diamine species which comprises at least one carbon dioxide derivative of hexane-1,6-diamine. Preferably, said liquid aqueous stream is obtained from a fermentation process.

Hexamethylenediamine, also referred to HMDA hereinbelow and further known as 1,6-di-aminohexane or 1,6-hexanediamine, is a chemical compound frequently used as a raw material, in particular for preparing polymers. The great majority of HMDA is employed for producing nylon 66 via condensation with adipic acid. Further, it is used for preparing hexa-methylene diisocyanate (HDI) via phosgenation which in turn is a monomer feedstock for preparing polyurethanes. Yet further, for example, HMDA serves as a cross-linking agent in epoxy resins.

Currently, the most used commercial process for HMDA manufacture proceeds via the hydrogenation of adiponitrile in ammonia, which is in turn produced by the hydrocyanation of butadiene. However, also bio-based routes are known and described, inter alia, in US 2017/0369913 A1. Usually, in such fermentation processes, carbon dioxide is used, in particular for adjusting the pH of the media involved. Thus, from said fermentation processes, aqueous solutions are obtained which contain carbon dioxide derivates and carbon dioxide adducts of HMDA such as carbonates and carbamates. However, in downstream applications where HMDA is used as a raw material, free HMDA is employed. Therefore, there is the need to provide an advantageous process for cleaving said derivatives and adducts to obtain a solution comprising HMDA in its free form. According to the present invention, such an advantageous process is provided.

Thus, the present invention is directed to a process for removing carbon dioxide from a liquid aqueous stream L₁ comprising at least one hexane-1,6-diamine species which comprises at least one carbon dioxide derivative of hexane-1,6-diamine, the process comprising
(i) providing the liquid aqueous stream L₁ exhibiting a carbon dioxide loading c_{L1}, wherein c_{L1} is defined as n_{L1}(CO₂)/n_{L1}(HMDA) with n_{L1}(CO₂) being the molar amount of carbon dioxide comprised in the at least one carbon dioxide derivative of hexane-1,6-diamine in L₁ and n_{L1}(HMDA) being the molar amount of the at least one hexane-1,6-diamine species in L₁, wherein 0.5≤c_{L1}≤2.5, wherein the liquid aqueous stream L₁ to be subjected to heating according to (ii) has a temperature T_{L1}<T according to (ii);
(ii) heating the liquid aqueous stream L₁ provided according to (i) to a temperature T≥90° C. at a pressure p≥0.5 bar(abs), obtaining a vapor stream V comprising carbon dioxide, and obtaining a liquid aqueous stream L₂ exhibiting a carbon dioxide loading c_{L2}, wherein c_{L2} is defined as n_{L2}(CO₂)/n_{L2}(HMDA) with n_{L2}(CO₂) being the molar amount of carbon dioxide comprised in the at least one carbon dioxide derivative of hexane-1,6-diamine in L₂ and n_{L2}(HMDA) being the molar amount of the at least one hexane-1,6-diamine species in L₂, wherein c_{L2}≤0.1.

The term “carbon dioxide derivative of hexane-1,6-diamine” as used herein comprises all conceivable carbonates and carbamates of HMDA. Preferably, this term comprises hexa-methylene-1,6-carbamate (H₂N—(CH₂)₆—NH—COO⁻), hexamethylene-1,6-dicarbamate (⁻OOC—HN—(CH₂)₆—NH—COO⁻), hexamethylene-1,6-carbamate zwitter-ion (⁺H₃N—(CH₂)₆—NH—COO⁻), he-xamethylene-1,6-carbonate (⁺H₃N—(CH₂)₆—NH₃⁺CO₃²⁻), hexamethylene-1,6-bi-carbonate (H₂N—(CH₂)₆—NH₃⁺HCO₃⁻), and hexamethylene-1,6-bis-bicarbonate (⁺H₃N—(CH₂)₆—NH₃⁺(HCO₃⁻)₂). Preferably, the counter-ion of hexamethylene-1,6-carbamate and hexamethylene-1,6-dicarbamate is either single-protonated or double-protonated HMDA. Preferably according to the present invention, from 80 to 100 mol-%, more preferably from 90 to 100 mol-%, more preferably from 95 to 100 mol-% of the at least one carbon dioxide derivative of hexane-1,6-diamine consist of one or more of hexamethylene-1,6-carbamate, hexamethylene-1,6-dicarbamate, hexamethylene-1,6-carbamate zwitter-ion, hexamethylene-1,6-carbonate, hexamethylene-1,6-bicarbonate, and hexamethylene-1,6-bis-bicarbonate.

Generally, the at least one hexane-1,6-diamine species referred to in the present invention comprises the at least one carbon dioxide derivative of hexane-1,6-diamine described above. Optionally, the at least one hexane-1,6-diamine species additionally comprises other hexane-1,6-diamine species, preferably hexane-1,6-diamine free base. According to an embodiment of the present invention, the at least one hexane-1,6-diamine species consists of the one or more carbon dioxide derivatives of hexane-1,6-diamine described above and hexane-1,6-diamine free base. Preferably from 90 to 100 mol-%, more from 95 to 100 mol-%, more preferably from 98 to 100 mol-% of the at least one hexane-1,6-diamine species consist of the at least one carbon dioxide derivative of hexane-1,6-diamine and optionally hexane-1,6-diamine free base.

The total concentration of the at least one carbon dioxide derivative of hexane-1,6-diamine in the liquid aqueous stream L₁ provided according to (i) is preferably in the range of from 3 to 30 weight-%, more preferably in the range of from 5 to 25 weight-%, more preferably in the range of from 7 to 15 weight-%, such as from 7 to 9 weight-% or from 9 to 11 weight-% or from 11 to 13 weight-% or from 13 to 15 weight-% based on the total weight of the stream L₁. The term “total concentration of the at least one carbon dioxide derivative of hexane-1,6-diamine” as used in this regard is to be understood as being calculated on a CO₂-free basis, i.e. the weight of each carbon dioxide derivative of hexane-1,6-diamine is expressed as the weight of HMDA free base, and the liquid aqueous stream L₁ is assumed to be free of CO₂.

Regarding the carbon dioxide loading c_{L1} according to (i), preferred ranges are 0.6≤c_{L1}≤2.0, more preferably 0.7≤c_{L1}≤1.6, such as 0.7≤c_{L1}≤1.0 or 1.0≤c_{L1}≤1.3 or 1.3≤c_{L1}≤1.6.

Generally, the liquid aqueous stream L₁ provided according to (i) may comprise, in addition to water, the at least carbon dioxide derivative of hexane-1,6-diamine and optionally hexane-1,6-diamine free base one or more further compounds wherein it is preferred that from 90 to 100 weight-%, preferably from 95 to 100 weight-%, more preferably from 98 to 100 weight-%, more preferably from 99 to 100 weight-% of the liquid aqueous stream L₁ provided according to (i) consist of water, the at least carbon dioxide derivative of hexane-1,6-diamine and optionally hexane-1,6-diamine free base Preferably, the liquid aqueous stream L₁ provided according to (i) has a pH of at least 6.

In particular in case the liquid aqueous stream L₁ is obtained from a fermentation process, it is preferred that it has a pH in the range of from 6 to 10, more preferably in the range of from 7 to 9.5, more preferably in the range of from 7.5 to 9, such as from 7.5 to 8 or from 8 to 8.5 of from 8.5 to 9.

According to (ii), the liquid aqueous stream L₁ provided according to (i) is heated to a temperature T of the liquid stream of at least 90° C. at a pressure p≥0.5 bar(abs) wherein T>T_{L1}. Preferably, T_{L1} is at least 10° C., more preferably at least 15° C., more preferably at least 20° C. Preferred ranges of T are, for example, from 10 to 50° C. or from 15 to 40° C. or from 20 to 30° C. Preferably, 10° C.≤T_{L1}<T, more preferably 15° C.≤T_{L1}<T, more preferably 20° C.≤T_{L1}<T.

According to (ii), the temperature T to which the liquid stream L₁ is heated in said column is preferably in the range of from 90 to 190° C., more preferably in the range of from 100 to 180° C., more preferably in the range of from 110 to 170° C. Thus, preferred range may be in the range of from 110 to 120° C. or from 120 to 130° C. or from 130 to 140° C. or from 140 to 150° C. or from 150 to 160° C. of from 160 to 170° C.

From step (ii) of the present invention, the liquid aqueous strip L₂ is obtained which contains a significantly reduced carbon dioxide loading since during (ii), the at least one carbon dioxide derivative of HMDA is cleaved, CO₂ is set free and removed from via the vapor stream V while an aqueous solution comprising HMDA free base is obtained as the stream L₂. Preferably, the carbon dioxide loading c_{L2} of the stream L₂ is at most 0.08, more preferably at most 0.06, more preferably at most 0.04, more preferably at most 0.03, more preferably at most 0.02. Therefore, preferably 0≤c_{L2}≤0.08, more preferably 0≤c_{L2}≤0.06, more preferably 0≤c_{L2}≤0.04, more preferably 0≤c_{L2}≤0.03, more preferably 0≤c_{L2}≤0.02.

According to a first embodiment of the present invention, the heating according to (ii) is carried out in a stripping column, wherein a stripping medium is used and wherein providing the liquid aqueous stream L₁ according to (i) comprises feeding the liquid aqueous stream L₁ into the stripping column, and wherein (ii) comprises
(ii.1) heating the liquid aqueous stream L₁ provided according to (i) in the stripping column to a temperature T of at least 90° C. at a pressure p of at least 0.5 bar(abs);
(ii.2) obtaining a vapor stream V at the top of the stripping column, the vapor stream V having a temperature T_{V} and comprising carbon dioxide, and removing said vapor stream V from the top of the stripping column;
(ii.3) obtaining the liquid aqueous stream L₂ at the bottoms of the stripping column, the liquid aqueous stream having a temperature T_{L2} and comprising hexane-1,6-diamine free base, and removing said liquid aqueous stream L₂ from the bottoms of the stripping column;
and wherein the process further comprises
(iii.1) subjecting the liquid aqueous stream L₂ obtained according to (ii.3) to evaporation in an evaporator E₁, obtaining an aqueous vapor stream V_{L2} having a temperature T_{VL2} and an aqueous liquid stream L₃ having a temperature T_{L3}, wherein preferably T_{VL2}=T_{L3};
(iii.2) feeding the aqueous vapor stream V_{L2} obtained according to (iii.1) back into the bottoms section of the stripping column.

No specific restrictions exist as far as the stripping medium is concerned. Preferably, the stripping medium comprises steam, wherein more preferably, from 90 to 100 weight-%, more preferably from 95 to 100 weight-%, more preferably from 99 to 100 weight-% of the stripping medium consist of steam. Hence, preferably from 0 to 10 weight-%, more preferably from 0 to 5 weight-%, more preferably from 0 to 1 weight-% of the stripping medium consist of one or more stripping media other than steam, preferably of one or more of nitrogen, air, and lean air. Thus, while other stripping media besides steam are conceivable, it is preferred that 100 weight-% of the stripping medium consists of steam. While this steam may be introduced into the stripping column from one or more suitable external sources, it is preferred that the steam is at least partially, preferably entirely obtained in situ in the stripping column by heating the liquid aqueous stream L₂ in the stripping column to the temperature T.

According to a preferred embodiment of the present invention, the liquid stream L₃ which is obtained in (iii.1) from the evaporator E₁ as described above is passed through a heat exchanger H₁. In this heat exchanger H₁, a part of the heat contained in L₃ is transferred to another stream, preferably to the stream L₁ which is thus suitably preheated in H₁ before it passed into the stripping column. Therefore, the process of the present invention preferably further comprises
(iii.3) passing the aqueous liquid stream L₃ obtained according to (iii.1) through a heat exchanger H₁, obtaining a stream L₄ having a temperature T_{L4} with T_{L4}<T_{L3};
(iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10}.

Preferably, the temperature difference ΔT_{H1}=T_{L10}−T_{L4}, i.e. the temperature difference between the temperature of L₁ after being passed through H₁ and the temperature of the stream L₄, i.e. the temperature of the stream L₃ after having transferred a part of the heat contained therein to L₁ in H₁, is at most 20 K, more preferably at most 15 K, more preferably at most 10 K, more preferably at most 5 K.

Regarding the vapor stream V obtained at the top of stripping column according to (ii.2), it preferably comprises water in addition to carbon dioxide. In order to separate the water from the carbon dioxide, it is preferred that the stream V is subjected to a suitable separation stage, preferably to condensation from which a carbon dioxide-depleted water stream and a water-depleted carbon dioxide stream are obtained. Therefore, the present process preferably further comprises, in the preferred case that the vapor stream V comprises water in addition to carbon dioxide,
(iv.1) subjecting the vapor stream V obtained according to (ii.2) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

Generally, it may be advantageous to operated the stripping column with internal reflux. According to this embodiment, the stream L_{V} obtained according to (iv.1) above is suitably divided, and a stream accordingly obtained is sent back to the stripping column, preferably into the top section of the stripping column. In this case, the process of the present invention preferably further comprises
(iv.2) dividing the liquid stream L_{V} obtained according to (iv.1) in two streams L_{V1} and L_{V2}, and feeding the stream L_{V1} back into the top section of the stripping column.

As far as the respective reflux ratio is concerned, no specific restrictions exist. Preferably, the reflux ratio is in the range of from 0.01:1 to 1:1, more preferably in the range of from 0.03:1 to 0.3:1, more preferably in the range of from 0.05:1 to 0.2:1.

According to a further embodiment of the present invention, the vapor stream V obtained at the top of the stripping column is used to transfer a part of its heat in a heat exchanger H₂ to another stream, preferably to aqueous liquid stream L₁ before it is passed into the stripping column. As far as this embodiment is concerned, it is preferred that this heat exchanger H₂ is arranged upstream of the heat exchanger H₁ according to (iii.4) described above, i.e. the stream L₁, prior to being passed into the stripping column, is preheated to a first increased temperature, and the resulting preheated stream is then further preheated to a second increased temperature which is higher than the first increased temperature. Yet further, according to this embodiment, it is preferred that the stream obtained from H₂ which has transferred part of its heat to L₁ is passed into a condenser from which a carbon dioxide-depleted water stream and a water-depleted carbon dioxide stream are obtained. Thus, the process of the present invention wherein the vapor stream V obtained according to (ii.2) further comprises water in addition to carbon dioxide, preferably further comprises
(iv.0) passing the vapor stream V, obtained according to (ii.2), through a heat exchanger H₂, obtaining a partially condensed stream VL having a temperature T_{HVL}<T_{V};
(iv.1) subjecting the stream VL obtained according to (iv.0) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
wherein the process more preferably further comprises passing the liquid aqueous stream L₁ having a temperature T_{L100}, prior to passing it through the heat exchanger H₁ according to (iii.4), through the heat exchanger H₂ according to (iv.0), obtaining the liquid aqueous stream L₁ having the temperature T_{L10} with T_{L10}>T_{L100}.

Preferably, the temperature difference ΔT_{H2}=T_{L100}−T_{HVL}, i.e. the temperature difference between the temperature of the L₁ after having being passed through H₂ and the temperature of the stream VL, i.e. the temperature of the stream V after having transferred a part of the heat contained therein to L₁ in H₂, is at most 20 K, more preferably at most 15 K, more preferably at most 10 K, more preferably at most 5 K.

According to the present invention, the latter embodiment may further exhibit a suitable internal reflux to the top of the stripping column. In this respect, it is preferred that the process further comprises, after (iv.0) and prior to (iv.1),
(iv.2) dividing the stream VL obtained according to (iv.0) in two streams VL₁ and VL₂, feeding the stream VL₁ back into the top section of the stripping column and subjecting the stream VL₂ as stream VL according to (iv.1) to condensation in the condenser C₁ according to (iv.2).

As far as the respective reflux ratio is concerned, no specific restrictions exist. Preferably, said reflux ratio is in the range of from 0.01:1 to 1:1, more preferably in the range of from 0.03:1 to 0.3:1, more preferably in the range of from 0.05:1 to 0.2:1.

According to a further embodiment of the present invention, the vapor stream obtained at the top of the stripping column is subjected to compression, and the respectively obtained compressed stream is used as a heating medium of the evaporator of stripping column. The thus obtained cooled compressed stream or a part thereof which leaves the evaporator is then preferably fed back to the top of the stripping column. In this case, the process of the present invention further comprises
(v.1) removing a vapor stream V_{K} having a temperature T_{VK} and comprising carbon dioxide and preferably water from the top of the stripping column, the vapor stream V_{K} preferably having the same chemical composition as the vapor stream V;
(v.2) passing the stream V_{K} removed from the top of the stripping column according to (v.1) through a compressor K₁, obtaining a compressed stream V_{K} having a temperature T_{CVK} with T_{CVK}>T_{VK};
(v.3) passing the compressed stream V_{K} as a heating medium through the evaporator E₁ according to (iii.1), obtaining a cooled compressed stream V_{K} comprising a liquid phase V_{K}(l) and optionally a vapor phase V_{K}(g);
(v.4) preferably feeding the liquid phase V_{K}(l) obtained according to (v.3) or a portion thereof into the top section of the stripping column.

As indicated in (v.3), the cooled compressed stream VK may contain, in addition to a liquid phase which may be fed back to the top of the column, a vapor phase. In this case, it may be preferred if the stream VK is subjected to a suitable gas-liquid separation stage. As far as the respectively obtained vapor phase is concerned, it may be preferred to combine it with another stream obtained in the process, preferably the vapor stream obtained from the compressor which is arranged downstream of the heat exchanger H2 as described above. Thus, the process may preferably further comprise
(v.5) combining the vapor phase V_{K}(g) obtained according to (v.3) with the vapor stream V_{V} obtained according to (iv.1).

According to a further embodiment, the process is designed in such a way that the stripping column is suitably combined with concentrating a carbon dioxide-depleted aqueous solution, which concentrating is carried out by evaporation. According to this embodiment, the stripping column is used essentially to strip off the carbon dioxide contained in the liquid stream L₁, and the respectively resulting aqueous stream which is depleted in carbon dioxide is then subjected to a downstream evaporation stage operated at reduced pressure compared to the stripping column. Advantageously, for this evaporation task, the vapor stream, or a part thereof, obtained from the top of the stripping column is used. Still more preferably, the steam which is obtained from said evaporation is used to preheat the liquid stream L₁ before it is passed into the stripping column. According to this process setup, a highly efficient and heat-integrated process is provided which leads to a concentrated aqueous solution comprising HMDA free base.

According to this embodiment which combines stripping and concentrating, the process preferably further comprises
(iii.3) passing the vapor stream V, obtained according to (ii.2), through an evaporator E₂, obtaining the stream V having a temperature T_{EV}<T_{V};
(iii.4) subjecting the aqueous liquid stream L₃ obtained according to (iii.1) to evaporation in the evaporator E₂ according to (iii.3), obtaining an aqueous vapor stream V_{L3} having a temperature T_{VL3} and an aqueous liquid stream L_{L3} having a temperature T_{LL3}, wherein preferably T_{VL3}=T_{LL3}.

Further preferably, this process comprises
(iii.5) passing the aqueous vapor stream VL₃ obtained according to (iii.4) through a heat exchanger H₁, obtaining an aqueous vapor stream V_{L3H} having a temperature T_{VL3H} with T_{VL3H}<T_{VL3};
(iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10}.

Still further preferably, this process comprises subjecting the aqueous vapor stream V_{L3H} obtained according to (iii.5) to condensation in a condenser C₂, obtaining a liquid aqueous stream.

As discussed hereinabove with respect to previous embodiment, also the process setup which combines stripping and concentrating may exhibit, as an additional feature, the condensation of the vapor stream obtained from the stripping column and, further preferably, the internal reflux using at least a part of the respectively obtained liquid stream. According to this embodiment, wherein the vapor stream V obtained according to
(iii.3) further comprises water in addition to carbon dioxide, the process preferably further comprises
(iv.1) subjecting the vapor stream V obtained according to (iii.3) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

Further preferably, this process comprises
(iv.2) dividing the liquid stream L_{V} obtained according to (iv.1) in two streams L_{V1} and L_{V2}, and feeding the stream L_{V1} back into the top section of the stripping column.

As far as the respective reflux ratio is concerned, no specific restrictions exist.

Preferably, the reflux ratio is in the range of from 0.01:1 to 1:1, more preferably in the range of from 0.03:1 to 0.3:1, more preferably in the range of from 0.05:1 to 0.2:1.

According to another preferred embodiment of the present invention, the aqueous solution being depleted in carbon dioxide and comprising HMDA free base for further downstream use is prepared making use of a multistage evaporation setup. According to this embodiment, step (ii) is carried out in an evaporation unit comprising n serially coupled evaporation apparatuses Eⱼ, j=1 . . . n with n>2, wherein an evaporation apparatus Eⱼ comprises heating means E_{Hj}, wherein an evaporation apparatus Eⱼ₊₁ is arranged downstream of an evaporation apparatus Eⱼ, wherein providing the liquid aqueous stream L₁ according to (i) comprises feeding the liquid aqueous stream L₁ for evaporation into the evaporation apparatus E₁;
wherein for j=1 . . . n, (ii) comprises
heating the liquid aqueous stream having been fed into the evaporation apparatus Eⱼ to a temperature Tⱼ at a pressure pⱼ in said evaporation apparatus Eⱼ, obtaining a vapor stream Vⱼ, the vapor stream Vⱼ having a temperature T_{Vj} and comprising carbon dioxide, and removing said vapor stream Vⱼ from the evaporation apparatus Eⱼ, and obtaining a liquid aqueous stream L₁ⱼ, the liquid aqueous stream L₁ⱼ having a temperature T_{L1j} and exhibiting a carbon dioxide loading c_{L1j}, wherein c_{L1j} is defined as n_{L1j}(CO₂)/n_{L1j}(HMDA) with n_{L1j}(CO₂) being the molar amount of carbon dioxide comprised in the at least one carbon dioxide derivative of hexane-1,6-diamine in L₁ⱼ and n_{L1j}(HMDA) being the molar amount of the at least one hexane-1,6-diamine species in L₁ⱼ, and removing said liquid aqueous stream L₁ⱼ from the evaporation apparatus Eⱼ; and
feeding the liquid aqueous stream L₁ⱼ removed from Eⱼ into the evaporation apparatus Eⱼ₊₁ for evaporation and passing the vapor stream V_{Lj} as a heat source through the heating means E_{Hj+1} of the evaporation apparatus Eⱼ₊₁, and removing a stream W_{Lj+1} from the heating means E_{Hj+1};
and wherein for j=n, (ii) comprises
heating the liquid aqueous stream having been fed into the evaporation apparatus Eₙ to a temperature Tₙ at a pressure pₙ in said evaporation apparatus Eₙ, obtaining a vapor stream Vₙ=V, the vapor stream V having a temperature T_{V} and comprising carbon dioxide, and removing said vapor stream V from the evaporation apparatus Eₙ; and obtaining a liquid aqueous stream L₁ₙ=L₂, the liquid aqueous stream L₂ having a temperature T_{L2} and exhibiting the carbon dioxide loading c_{L2}, and removing said liquid aqueous stream L₂ from the evaporation apparatus Eₙ;
wherein one parameter pair (Tⱼ;pⱼ) is the parameter pair (T;p) as defined in claim 1 with a temperature T≥90° C. and a pressure p≥0.5 bar(abs).

According to a preferred embodiment regarding the multistage evaporation design, the parameter pair (T₁;p₁) which is realized in the first evaporator E₁ is the parameter pair (T;p) as defined hereinabove with a temperature T≥90° C. and a pressure p≥0.5 bar(abs). Preferably, the temperature T is in the range of from 90 to 190° C., more preferably in the range of from 110 to 190° C., more preferably in the range of from 130 to 190° C., such as in the range of from 130 to 150° C. or from 150 to 170° C. of from 170 to 190° C.

Regarding the number of serially coupled evaporation stages, n, it is preferred that n=7; more preferably that n=6; more preferably that n=5, more preferably that n=4, more preferably that n=3 or n=2.

Preferably, a given evaporator is operated at a pressure which is lower than that of the respectively previous evaporator, i.e. it is preferred that for j=1 . . . n−1, pⱼ₊₁<pⱼ and Tⱼ₊₁<Tⱼ.

Yet further, a given evaporator is preferable operated so that the temperature difference between the temperature of the vapor phase obtained from said evaporator and the temperature of the liquid stream obtained from the respectively next evaporator is in a certain range. Specifically, it is preferred that for j=1 . . . n−1, ΔT_{Ej+1}=T_{Vj}−T_{L1j+1} is in the range of from 5 to 10 K.

In particular, for n=2, it is preferred that
T₁ is in the range of from 90 to 190° C., preferably in the range of from 110 to 190° C., more preferably in the range of from 130 to 190° C.;
T₂ is in the range of from 45 to 170° C., preferably in the range of from 60 to 170° C., more preferably in the range of from 90 to 170° C.

In particular, for n=3, it is preferred that
T₁ is in the range of from 90 to 190° C., preferably in the range of from 110 to 190° C., more preferably in the range of from 130 to 190° C.;
T₂ is in the range of from 50 to 160° C., preferably in the range of from 70 to 160° C., more preferably in the range of from 90 to 160° C.;
T₃ is in the range of from 35 to 140° C., preferably in the range of from 50 to 140° C., more preferably in the range of from 70 to 140° C.

According to this multistage evaporation design, the carbon dioxide loading of a liquid stream obtained from a given evaporator is lower than the carbon dioxide loading of the liquid stream obtained from the previous evaporator so that, from the last evaporator, the liquid stream with the lowest carbon dioxide loading is obtained, i.e. that for j=1 . . . n−1, c_{L1j+1}<c_{L1j}.

Preferably, in case the vapor stream V obtained from the last evaporator further comprises water in addition to carbon dioxide, the process further comprises subjecting the vapor stream V to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

As for certain embodiments discussed hereinabove in the context of the stripping column designs, also the multi-evaporation design allows for providing a heat integration including a pre-heating of the stream L1 which is subjected to the first evaporator. According to this setup, the process preferably comprises passing the vapor stream V, obtained according to (ii.2), through a heat exchanger H₁, obtaining an optionally partially condensed stream VL having a temperature T_{VL}<T_{V}, wherein the process further comprises passing the liquid aqueous stream L₁ having a temperature T_{L10} through the heat exchanger H₁ obtaining the liquid aqueous stream L₁ having the temperature T_{L1} with T_{L1}>T_{L10}.

Preferably, the temperature difference ΔT_{H1}=T_{L10}−T_{VL} is at most 20 K, more preferably at most 10 K, more preferably at most 5 K.

As far as the first evaporator E₁ is concerned, the heating medium used for evaporation purposes is fed from an external source. In particular, the process further comprises passing a stream S₁₁ having a temperature T_{S11} as a heat source through the heating means E_{H1} of the evaporation apparatus E₁, and removing a stream S₁₂ from the heating means E_{H1}. Preferably, ΔT_{E1}=T_{S12}−T_{L11} is in the range of from 5 to 10 K. More preferably, the stream S₁₁ is a vapor stream, and stream S₁₂ is steam condensate.

Generally, according to the present invention, the feed stream liquid aqueous stream L1 can be obtained from any suitable source. According to a preferred embodiment, the stream L₁ which comprises at least one hexane-1,6-diamine species which in turn comprises at least one carbon dioxide derivative of hexane-1,6-diamine is obtainable or obtained by a fermentation process. Therefore, the present invention is further directed to a process as described above wherein providing the liquid aqueous stream L₁ according to (i) comprises preparing the stream L₁ in a fermentation process.

As far as such fermentation process is concerned, it is preferred that a genetically engineered microorganism is cultured or grown in a suitable reaction vessel in a suitable culture or fermentation medium which comprises a nitrogen source and a carbon source. In the course of said fermentation of the genetically engineered microorganism wherein HMDA is prepared, carbon dioxide is employed to adjust and to control the pH of the medium. As far as said carbon dioxide is concerned, it is possible to produce same either metabolically by the microorganism, or artificially. Alternatively, it can be added from a suitable external source. Preferably, the growth condition of the microorganism and the carbon dioxide concentration are controlled such that the pH is maintained at a desired value for specific periods of time during fermentation. Usually, during the fermentation process, the pH will increase from a value in the range of from about 6.5 to about 7.5 to a pH of about 8.5 since, e.g., a buffer is formed by HMDA and carbon dioxide. Once the fermentation is complete, i.e. the at least one hexane-1,6-diamine species is formed, the cells may be removed, for example by suitable filtration to separate the crude aqueous solution from undesired by-products comprised in the retentate. The aqueous fermentation solution which is obtained by such suitable filtering is referred to herein as the liquid aqueous stream L₁.

Preferably, according to the present invention, the ultimately obtained stream L₂ or a downstream stream thereof, as described hereinabove as the stream L₃, the stream L₄ or the stream L_{L3}, said stream being depleted in CO₂ and containing the free HMDA, are preferably subjected to a suitable post-treatment, more preferably to a suitable separation stage wherein the free HMDA is separated from said stream. Preferably, said separation stage comprises an extraction stage wherein said stream is brought into contact with a suitable organic extraction solvent. More preferably, said separation stage further comprises a distillation stage downstream of said extraction stage wherein is said distillation stage, the organic effluent stream obtained from extraction is suitable distilled to separate the HMDA from the extraction solvent. It may be preferred to recycle the thus separated extraction solvent back into the extraction stage.

Therefore, according to the present invention, the stream L₂ or a downstream stream obtained therefrom is subjected to extraction, wherein the downstream stream obtained from L₂ is the stream L₃ as defined above, the stream L₄ as defined above, or the stream L_{L3} as defined above.

The thus separated HMDA can be used as such or be subjected to further purification, for example in a further downstream distillation stage. The respectively obtained HMDA can then be used, for example, as a starting material for preparing polyamides such as nylon, polyureas, isocyanates such as hexamethylene diisocyanates, polyurethanes, and copolymers of one or more thereof, and for a (semiquantitative) detection reaction for certain sugars such as disaccharides—like lactose, maltose, cellobiose, lactulose or maltulose.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.
1. A process for removing carbon dioxide from a liquid aqueous stream L₁ comprising at least one hexane-1,6-diamine species which comprises at least one carbon dioxide derivative of hexane-1,6-diamine, the process comprising
   (i) providing the liquid aqueous stream L₁ exhibiting a carbon dioxide loading c_{L1}, wherein c_{L1} is defined as n_{L1}(CO₂)/n_{L1}(HMDA) with n_{L1}(CO₂) being the molar amount of carbon dioxide comprised in the at least one carbon dioxide derivative of hexane-1,6-diamine in L₁ and n_{L1}(HMDA) being the molar amount of the at least one hexane-1,6-diamine species in L₁, wherein 0.5≤c_{L1}≤2.5, wherein the liquid aqueous stream L₁ to be subjected to heating according to (ii) has a temperature T_{L1}<T according to (ii);
   (ii) heating the liquid aqueous stream L₁ provided according to (i) to a temperature T≥90° C. at a pressure p≥0.5 bar(abs), obtaining a vapor stream V comprising carbon dioxide, and obtaining a liquid aqueous stream L₂ exhibiting a carbon dioxide loading c_{L2}, wherein c_{L2} is defined as n_{L2}(CO₂)/n_{L2}(HMDA) with n_{L2}(CO₂) being the molar amount of carbon dioxide comprised in the at least one carbon dioxide derivative of hexane-1,6-diamine in L₂ and n_{L2}(HMDA) being the molar amount of the at least one hexane-1,6-diamine species in L₂, wherein c_{L2}≤0.1.
2. The process of embodiment 1, wherein from 80 to 100 mol-%, preferably from 90 to 100 mol-%, more preferably from 95 to 100 mol-% of the at least one carbon dioxide derivative of hexane-1,6-diamine consist of one or more of hexamethylene-1,6-carbamate, hexamethylene-1,6-dicarbamate, hexamethylene-1,6-carbamate zwitter-ion, hexamethylene-1,6-carbonate, hexamethylene-1,6-bicarbonate, and hexamethylene-1,6-bis-bicarbonate.
3. The process of embodiment 1 or 2, wherein from 90 to 100 mol-%, preferably from 95 to 100 mol-%, more preferably from 98 to 100 mol-% of the at least one hexane-1,6-diamine species consist of the at least one carbon dioxide derivative of hexane-1,6-diamine and optionally hexane-1,6-diamine free base.
4. The process of any one of embodiments 1 to 3, wherein the total concentration of the at least one carbon dioxide derivative of hexane-1,6-diamine in the liquid aqueous stream L₁ provided according to (i) is in the range of from 3 to 30 weight-%, preferably in the range of from 5 to 25 weight-%, more preferably in the range of from 7 to 15 weight-%, based on the total weight of the stream L₁.
5. The process of any one of embodiments 1 to 4, wherein 0.6≤c_{L1}≤2.0, preferably 0.7≤c_{L1}≤1.6.
6. The process of any one of embodiments 1 to 5, wherein from 90 to 100 weight-%, preferably from 95 to 100 weight-%, more preferably from 99 to 100 weight-% of the liquid aqueous stream L₁ provided according to (i) consist of water, the at least carbon dioxide derivative of hexane-1,6-diamine and optionally hexane-1,6-diamine free base.
7. The process of any one of embodiments 1 to 6, wherein the liquid aqueous stream L₁ provided according to (i) has, at a temperature of L₁ of 25° C., a pH in the range of from 6 to 10, preferably in the range of from 7 to 9.5, more preferably in the range of from 7.5 to 9.
8. The process of any one of embodiments 1 to 7, wherein 10° C.≤T_{L1}<T, preferably 15° C.≤T_{L1}<T, more preferably 20° C.≤T₁<T.
9. The process of any one of embodiments 1 to 8, wherein the temperature T according to (ii) is in the range of from 90 to 190° C., preferably in the range of from 100 to 180° C., more preferably in the range of from 110 to 170° C.
10. The process of any one of embodiments 1 to 9, wherein c_{L2}≤0.08, preferably c_{L2}≤0.06, more preferably c_{L2}≤0.04, more preferably c_{L2}≤0.02.
11. The process of any one of embodiments 1 to 10, wherein step (ii) is carried out in a stripping column using a stripping medium, wherein providing the liquid aqueous stream L₁ according to (i) comprises feeding the liquid aqueous stream L₁ into the stripping column, and wherein (ii) comprises
   (ii.1) heating the liquid aqueous stream L₁ provided according to (i) in the stripping column to a temperature T of at least 90° C. at a pressure p of at least 0.5 bar(abs);
   (ii.2) obtaining a vapor stream V at the top of the stripping column, the vapor stream V having a temperature T_{V} and comprising carbon dioxide, and removing said vapor stream V from the top of the stripping column;
   (ii.3) obtaining the liquid aqueous stream L₂ at the bottoms of the stripping column, the liquid aqueous stream having a temperature T_{L2} and comprising hexane-1,6-diamine free base, and removing said liquid aqueous stream L₂ from the bottoms of the stripping column;
   the process further comprising
   (iii.1) subjecting the liquid aqueous stream L₂ obtained according to (ii.3) to evaporation in an evaporator E₁, obtaining an aqueous vapor stream V_{L2} having a temperature T_{VL2} and an aqueous liquid stream L₃ having a temperature T_{L3}, wherein preferably T_{VL2}=T_{L3};
   (iii.2) feeding the aqueous vapor stream V₂ obtained according to (iii.1) back into the bottoms section of the stripping column.
12. The process of embodiment 11, wherein the stripping medium comprises steam, wherein preferably from 90 to 100 weight-%, more preferably from 95 to 100 weight-%, more preferably from 99 to 100 weight-% of the stripping medium consist of steam.
13. The process of embodiment 12, wherein the steam is at least partially, preferably entirely obtained in situ in the stripping column by heating the liquid aqueous stream L₁ in the stripping column to the temperature T.
14. The process of any one of embodiments 11 to 13, wherein from 0 to 10 weight-%, preferably from 0 to 5 weight-%, more preferably from 0 to 1 weight-% of the stripping medium consist of one or more of nitrogen, air, and lean air.
15. The process of any one of embodiments 11 to 14, further comprising
   (iii.3) passing the aqueous liquid stream L₃ obtained according to (iii.1) through a heat exchanger H₁, obtaining a stream L₄ having a temperature T_{L4} with T_{L4}<T_{L3};
   (iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10}.
16. The process of embodiment 15, wherein according to (iii.4), ΔT_{H1}=T_{L10}−T_{L4} and wherein ΔT_{H1}≤20 K, preferably ΔT_{H1}≤10 K, more preferably ΔT_{H1}≤5 K.
17. The process of any one of embodiments 11 to 16, wherein the vapor stream V obtained according to (ii.2) further comprises water in addition to carbon dioxide, the process further comprising
   (iv.1) subjecting the vapor stream V obtained according to (ii.2) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.
18. The process of embodiment 17, further comprising
   (iv.2) dividing the liquid stream L_{V} obtained according to (iv.1) in two streams L_{V1} and L_{V2}, and feeding the stream L_{V1} back into the top section of the stripping column.
19. The process of embodiment 18, wherein the reflux ratio is in the range of from 0.01:1 to 1:1, preferably in the range of from 0.03:1 to 0.3:1, more preferably in the range of from 0.05:1 to 0.2:1.
20. The process of any one of embodiments 11 to 16, wherein the vapor stream V obtained according to (ii.2) further comprises water in addition to carbon dioxide, the process further comprising
   (iv.0) passing the vapor stream V, obtained according to (ii.2), through a heat exchanger H₂, obtaining a partially condensed stream VL having a temperature T_{HVL}<T_{V};
   (iv.1) subjecting the stream VL obtained according to (iv.0) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
   wherein the process further comprises passing the liquid aqueous stream L₁ having a temperature T_{L100}, prior to passing it through the heat exchanger H₁ according to (iii.4), through the heat exchanger H₂ according to (iv.0), obtaining the liquid aqueous stream L₁ having the temperature T_{L10} with T_{L10}>T_{L100}.
21. The process of embodiment 20, wherein ΔT_{LH2}=T_{L100}−T_{HVL} and wherein ΔT_{H2}≤20 K, preferably ΔT_{H1}≤10 K, more preferably ΔT_{H1}≤5 K.
22. The process of embodiment 20 or 21, further comprising, after (iv.0) and prior to (iv.1),
   (iv.2) dividing the stream VL obtained according to (iv.0) in two streams VL₁ and VL₂, feeding the stream VL₁ back into the top section of the stripping column and subjecting the stream VL₂ as stream VL according to (iv.1) to condensation in the condenser C₁ according to (iv.2).
23. The process of embodiment 22, wherein the reflux ratio is in the range of from 0.01:1 to 1:1, preferably in the range of from 0.03:1 to 0.3:1, more preferably in the range of from 0.05:1 to 0.2:1.
24. The process of any one of embodiments 11 to 23, further comprising
   (v.1) removing a vapor stream V_{K} having a temperature T_{VK} and comprising carbon dioxide and preferably water from the top of the stripping column, the vapor stream V_{K} preferably having the same chemical composition as the vapor stream V;
   (v.2) passing the stream V_{K} removed from the top of the stripping column according to (v.1) through a compressor K₁, obtaining a compressed stream V_{K} having a temperature T_{CVK} with T_{CVK}>T_{VK};
   (v.3) passing the compressed stream V_{K} as a heating medium through the evaporator E₁ according to (iii.1), obtaining a cooled compressed stream V_{K} comprising a liquid phase V_{K}(l) and optionally a vapor phase V_{K}(g);
   (v.4) preferably feeding the liquid phase V_{K}(l) obtained according to (v.3) or a portion thereof into the top section of the stripping column.
25. The process of embodiment 24 insofar as embodiment 24 is dependent on embodiment 17, the process further comprising
   (v.5) combining the vapor phase V_{K}(g) obtained according to (v.3) with the vapor stream V_{V} obtained according to (iv.1).
26. The process of any one of embodiments 11 to 14, further comprising
   (iii.3) passing the vapor stream V, obtained according to (ii.2), through an evaporator E₂, obtaining the stream V having a temperature T_{EV}<T_{V};
   (iii.4) subjecting the aqueous liquid stream L₃ obtained according to (iii.1) to evaporation in the evaporator E₂ according to (iii.3), obtaining an aqueous vapor stream V_{L3} having a temperature T_{VL3} and an aqueous liquid stream L_{L3} having a temperature T_{LL3}, wherein preferably T_{VL3}=T_{LL3}.
27. The process of embodiment 26, further comprising
   (iii.5) passing the aqueous vapor stream V_{L3} obtained according to (iii.4) through a heat exchanger H₁, obtaining an aqueous vapor stream V_{L3H} having a temperature T_{VL3H} with T_{VL3H}<T_{VL3};
   (iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10}.
28. The process of embodiment 27, further comprising subjecting the aqueous vapor stream V_{L3H} obtained according to (iii.5) to condensation in a condenser C₂, obtaining a liquid aqueous stream.
29. The process of any one of embodiments 26 to 28, wherein the vapor stream V obtained according to (iii.3) further comprises water in addition to carbon dioxide, the process further comprising
   (iv.1) subjecting the vapor stream V obtained according to (iii.3) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.
30. The process of embodiment 29, further comprising
   (iv.2) dividing the liquid stream L_{V} obtained according to (iv.1) in two streams L_{V1} and L_{V2}, and feeding the stream L_{V1} back into the top section of the stripping column.
31. The process of embodiment 30, wherein the reflux ratio is in the range of from 0.01:1 to 1:1, preferably in the range of from 0.03:1 to 0.3:1, more preferably in the range of from 0.05:1 to 0.2:1.
32. The process of any one of embodiments 1 to 10, wherein step (ii) is carried out in an evaporation unit comprising n serially coupled evaporation apparatuses Eⱼ, j=1 . . . n with n≥2, wherein an evaporation apparatus Eⱼ comprises heating means E_{Hj}, wherein an evaporation apparatus Eⱼ₊₁ is arranged downstream of an evaporation apparatus Eⱼ, wherein providing the liquid aqueous stream L₁ according to (i) comprises feeding the liquid aqueous stream L₁ for evaporation into the evaporation apparatus E₁;
   wherein for j=1 . . . n, (ii) comprises
   heating the liquid aqueous stream having been fed into the evaporation apparatus Eⱼ to a temperature Tⱼ at a pressure pⱼ in said evaporation apparatus Eⱼ, obtaining a vapor stream Vⱼ, the vapor stream Vⱼ having a temperature T_{Vj} and comprising carbon dioxide, and removing said vapor stream Vⱼ from the evaporation apparatus Eⱼ, and obtaining a liquid aqueous stream L₁ⱼ, the liquid aqueous stream L₁ⱼ having a temperature T_{L1j} and exhibiting a carbon dioxide loading c_{L1j}, wherein c_{L1j} is defined as n_{L1}(CO₂)/n_{L1j}(HMDA) with n_{L1j}(CO₂) being the molar amount of carbon dioxide comprised in the at least one carbon dioxide derivative of hexane-1,6-diamine in L₁ⱼ and n_{L1j}(HMDA) being the molar amount of the at least one hexane-1,6-diamine species in L₁ⱼ, and removing said liquid aqueous stream L₁ⱼ from the evaporation apparatus Eⱼ; and
   feeding the liquid aqueous stream L₁ⱼ removed from Eⱼ into the evaporation apparatus Eⱼ₊₁ for evaporation and passing the vapor stream V_{Lj} as a heat source through the heating means Eₕⱼ₊₁ of the evaporation apparatus Eⱼ₊₁, and removing a stream W_{Lj+1} from the heating means E_{Hj+1};
   and wherein for j=n, (ii) comprises
   heating the liquid aqueous stream having been fed into the evaporation apparatus Eₙ to a temperature Tₙ at a pressure pₙ in said evaporation apparatus Eₙ, obtaining a vapor stream Vₙ=V, the vapor stream V having a temperature T_{V} and comprising carbon dioxide, and removing said vapor stream V from the evaporation apparatus Eₙ; and obtaining a liquid aqueous stream L₁ₙ=L₂, the liquid aqueous stream L₂ having a temperature T_{L2} and exhibiting the carbon dioxide loading c_{L2}, and removing said liquid aqueous stream L₂ from the evaporation apparatus Eₙ; wherein one parameter pair (Tⱼ;pⱼ) is the parameter pair (T;p) as defined in claim 1 with a temperature T 90° C. and a pressure p 0.5 bar(abs).
33. The process of embodiment 32, wherein the parameter pair (T₁;p₁) is the parameter pair (T;p) as defined in embodiment 1 with a temperature T≥90° C. and a pressure p≥0.5 bar(abs).
34. The process of embodiment 33, wherein T is in the range of from 90 to 190° C., preferably in the range of from 110 to 190° C., more preferably in the range of from 130 to 190° C.
35. The process of any one of embodiments 32 to 34, wherein n=7; preferably wherein n=6; more preferably wherein n=5, more preferably wherein n=4, more preferably wherein n=3 or n=2.
36. The process of any one of embodiments 32 to 35, wherein for j=1 . . . n−1, pⱼ₊₁<pⱼ and Tⱼ₊₁<Tⱼ.
37. The process of embodiment 36, wherein for j=1 . . . n−1, ΔT_{Ej+1}=T_{Vj}−T_{L1j+1} is in the range of from 5 to 10 K.
38. The process of any one of embodiments 32 to 37, wherein n=2 and wherein
   T₁ is in the range of from 90 to 190° C., preferably in the range of from 110 to 190° C., more preferably in the range of from 130 to 190° C.;
   T₂ is in the range of from 45 to 170° C., preferably in the range of from 60 to 170° C., more preferably in the range of from 90 to 170° C.
39. The process of any one of embodiments 32 to 37, wherein n=3 and wherein
   T₁ is in the range of from 90 to 190° C., preferably in the range of from 110 to 190° C., more preferably in the range of from 130 to 190° C.;
   T₂ is in the range of from 50 to 160° C., preferably in the range of from 70 to 160° C., more preferably in the range of from 90 to 160° C.;
   T₃ is in the range of from 35 to 140° C., preferably in the range of from 50 to 140° C., more preferably in the range of from 70 to 140° C.
40. The process of any one of embodiments 32 to 39, wherein for j=1 . . . n−1, c_{L1j+1}<c_{L1j}.
41. The process of any one of embodiments 32 to 40, wherein the vapor stream V further comprises water in addition to carbon dioxide, the process further comprising subjecting the vapor stream V to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.
42. The process of any one of embodiments 32 to 40, further comprising passing the vapor stream V, obtained according to (ii.2), through a heat exchanger H₁, obtaining an optionally partially condensed stream VL having a temperature T_{VL}<T_{V}, wherein the process further comprises passing the liquid aqueous stream L₁ having a temperature T_{L10} through the heat exchanger H₁ obtaining the liquid aqueous stream L₁ having the temperature T_{L1} with T_{L1}>T_{L10}.
43. The process of embodiment 42, wherein ΔT_{H1}=T_{L10}−T_{VL} and wherein ΔT_{H1}≤20 K, preferably ΔT_{H1}<10 K, more preferably ΔT_{H1}≤5 K.
44. The process of any one of embodiments 32 to 43, further comprising passing a stream S₁₁ having a temperature T_{S11} as a heat source through the heating means E_{H1} of the evaporation apparatus E₁, and removing a stream S₁₂ from the heating means E_{H1}.
45. The process of embodiment 44, wherein ΔT_{E1}=T_{S12}−T_{L11} is in the range of from 5 to 10 K.
46. The process of embodiment 44 or 45, wherein the stream S₁₁ is a vapor stream.
47. The process of any one of embodiments 1 to 46, wherein the liquid aqueous stream L1 is obtained from a fermentation process.
48. The process of any one of embodiments 1 to 47, wherein the stream L₂ or a downstream stream obtained therefrom is subjected to extraction.
49. The process of embodiment 48, wherein the downstream stream obtained from L₂ is the stream L₃ as defined in embodiment 11, the stream L₄ as defined in embodiment 8, or the stream L₃ as defined in embodiment 26.
50. A process for removing carbon dioxide from a liquid aqueous stream L₁ comprising at least one hexane-1,6-diamine species which comprises at least one carbon dioxide derivative of hexane-1,6-diamine, the process comprising
   (i) providing the liquid aqueous stream L₁ exhibiting a carbon dioxide loading c_{L1}, wherein c_{L1} is defined as n_{L1}(CO₂)/n_{L1}(HMDA) with n_{L1}(CO₂) being the molar amount of carbon dioxide comprised in the at least one carbon dioxide derivative of hexane-1,6-diamine in L₁ and n_{L1}(HMDA) being the molar amount of the at least one hexane-1,6-diamine species in L₁, wherein 0.5≤c_{L1}≤2.5, wherein the liquid aqueous stream L₁ to be subjected to heating according to (ii) has a temperature T_{L1}<T according to (ii);
   (ii) heating the liquid aqueous stream L₁ provided according to (i) to a temperature T≥90° C. at a pressure p≥0.5 bar(abs), obtaining a vapor stream V comprising carbon dioxide, and obtaining a liquid aqueous stream L₂ exhibiting a carbon dioxide loading c_{L2}, wherein c_{L2} is defined as n_{L2}(CO₂)/n_{L2}(HMDA) with n_{L2}(CO₂) being the molar amount of carbon dioxide comprised in the at least one carbon dioxide derivative of hexane-1,6-diamine in L₂ and n_{L2}(HMDA) being the molar amount of the at least one hexane-1,6-diamine species in L₂, wherein c_{L2}≤0.1.
   wherein step (ii) is carried out in a stripping column using a stripping medium, wherein providing the liquid aqueous stream L₁ according to (i) comprises feeding the liquid aqueous stream L₁ into the stripping column, and wherein (ii) comprises
   (ii.1) heating the liquid aqueous stream L₁ provided according to (i) in the stripping column to a temperature T of at least 90° C. at a pressure p of at least 0.5 bar(abs);
   (ii.2) obtaining a vapor stream V at the top of the stripping column, the vapor stream V having a temperature T_{V} and comprising carbon dioxide, and removing said vapor stream V from the top of the stripping column;
   (ii.3) obtaining the liquid aqueous stream L₂ at the bottoms of the stripping column, the liquid aqueous stream having a temperature T_{L2} and comprising hexane-1,6-diamine free base, and removing said liquid aqueous stream L₂ from the bottoms of the stripping column;
   the process further comprising
   (iii.1) subjecting the liquid aqueous stream L₂ obtained according to (ii.3) to evaporation in an evaporator E₁, obtaining an aqueous vapor stream V_{L2} having a temperature T_{VL2} and an aqueous liquid stream L₃ having a temperature T_{L3}, wherein preferably T_{VL2}=T_{L3};
   (iii.2) feeding the aqueous vapor stream V₂ obtained according to (iii.1) back into the bottoms section of the stripping column;
   wherein the stripping medium preferably comprises steam, wherein more preferably from 90 to 100 weight-%, more preferably from 95 to 100 weight-%, more preferably from 99 to 100 weight-% of the stripping medium consist of steam, and
   wherein the steam is preferably at least partially, more preferably entirely obtained in situ in the stripping column by heating the liquid aqueous stream L₁ in the stripping column to the temperature T.
51. The process of embodiment 50, further comprising
   (iii.3) passing the aqueous liquid stream L₃ obtained according to (iii.1) through a heat exchanger H₁, obtaining a stream L₄ having a temperature T_{L4} with T_{L4}<T_{L3};
   (iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10};
   wherein according to (iii.4), ΔT_{H1}=T_{L10}−T_{L4} and wherein preferably ΔT_{H1}≤20 K,
   more preferably ΔT_{H1}≤10 K, more preferably ΔT_{H1}≤5 K.
52. The process of embodiment 50 or 51, wherein the vapor stream V obtained according to (ii.2) further comprises water in addition to carbon dioxide, the process further comprising
   (iv.1) subjecting the vapor stream V obtained according to (ii.2) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
   wherein the process preferably further comprises
   (iv.2) dividing the liquid stream L_{V} obtained according to (iv.1) in two streams L_{V1} and L_{V2}, and feeding the stream L_{V1} back into the top section of the stripping column, wherein the reflux ratio is preferably in the range of from 0.01:1 to 1:1, more preferably in the range of from 0.03:1 to 0.3:1, more preferably in the range of from 0.05:1 to 0.2:1.
53. The process of embodiment 50 or 51, wherein the vapor stream V obtained according to (ii.2) further comprises water in addition to carbon dioxide, the process further comprising
   (iv.0) passing the vapor stream V, obtained according to (ii.2), through a heat exchanger H₂, obtaining a partially condensed stream VL having a temperature T_{HVL}<T_{V};
   (iv.1) subjecting the stream VL obtained according to (iv.0) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
   wherein the process further comprises passing the liquid aqueous stream L₁ having a temperature T_{L100}, prior to passing it through the heat exchanger H₁ according to (iii.4), through the heat exchanger H₂ according to (iv.0), obtaining the liquid aqueous stream L₁ having the temperature T_{L10} with T_{L10}>T_{L100}.
   wherein ΔT_{H2}=T_{L100}−T_{HVL} and wherein preferably ΔT_{H2}≤20 K, more preferably ΔT_{H1}≤10 K, more preferably ΔT_{H1}≤5 K.
54. The process of embodiment 53, further comprising, after (iv.0) and prior to (iv.1),
   (iv.2) dividing the stream VL obtained according to (iv.0) in two streams VL₁ and VL₂, feeding the stream VL₁ back into the top section of the stripping column and subjecting the stream VL₂ as stream VL according to (iv.1) to condensation in the condenser C₁ according to (iv.2);
   wherein the reflux ratio is preferably in the range of from 0.01:1 to 1:1, more
   preferably in the range of from 0.03:1 to 0.3:1, more preferably in the range of from 0.05:1 to 0.2:1.
55. The process of any one of embodiments 50 to 54, further comprising
   (v.1) removing a vapor stream V_{K} having a temperature T_{VK} and comprising carbon dioxide and preferably water from the top of the stripping column, the vapor stream V_{K} preferably having the same chemical composition as the vapor stream V;
   (v.2) passing the stream V_{K} removed from the top of the stripping column according to (v.1) through a compressor K₁, obtaining a compressed stream V_{K} having a temperature T_{CVK} with T_{CVK}>T_{LVK};
   (v.3) passing the compressed stream V_{K} as a heating medium through the evaporator E₁ according to (iii.1), obtaining a cooled compressed stream V_{K} comprising a liquid phase V_{K}(l) and optionally a vapor phase V_{K}(g);
   (v.4) preferably feeding the liquid phase V_{K}(l) obtained according to (v.3) or a portion thereof into the top section of the stripping column.
56. The process of embodiment 50, further comprising
   (iii.3) passing the vapor stream V, obtained according to (ii.2), through an evaporator E₂, obtaining the stream V having a temperature T_{LEV}<T_{V};
   (iii.4) subjecting the aqueous liquid stream L₃ obtained according to (iii.1) to evaporation in the evaporator E₂ according to (iii.3), obtaining an aqueous vapor stream V_{L3} having a temperature T_{VL3} and an aqueous liquid stream L_{L3} having a temperature T_{LL3}, wherein preferably T_{VL3}=T_{LL3};
   the process preferably further comprising
   (iii.5) passing the aqueous vapor stream V_{L3} obtained according to (iii.4) through a heat exchanger H₁, obtaining an aqueous vapor stream V_{L3H} having a temperature T_{VL3H} with T_{VL3H}<T_{VL3};
   (iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10};
   wherein the aqueous vapor stream V_{L3H} obtained according to (iii.5) is preferably subjected to condensation in a condenser C₂, obtaining a liquid aqueous stream.
57. The process of embodiment 56, wherein the vapor stream V obtained according to
   (iii.3) further comprises water in addition to carbon dioxide, the process further comprising
   (iv.1) subjecting the vapor stream V obtained according to (iii.3) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
   the process preferably further comprising
   (iv.2) dividing the liquid stream L_{V} obtained according to (iv.1) in two streams L_{V1} and L_{V2}, and feeding the stream L_{V1} back into the top section of the stripping column;
   wherein the reflux ratio is preferably in the range of from 0.01:1 to 1:1, more preferably in the range of from 0.03:1 to 0.3:1, more preferably in the range of from 0.05:1 to 0.2:1.
58. The process of any one of embodiments 50 to 57, wherein from 80 to 100 mol-%, preferably from 90 to 100 mol-%, more preferably from 95 to 100 mol-% of the at least one carbon dioxide derivative of hexane-1,6-diamine consist of one or more of hexamethylene-1,6-carbamate, hexamethylene-1,6-dicarbamate, hexamethylene-1,6-carbamate zwitterion, hexamethylene-1,6-carbonate, hexamethylene-1,6-bicarbonate, and hexamethy-lene-1,6-bis-bicarbonate;
   wherein preferably from 90 to 100 mol-%, more preferably from 95 to 100 mol-%, more preferably from 98 to 100 mol-% of the at least one hexane-1,6-diamine species consist of the at least one carbon dioxide derivative of hexane-1,6-diamine and optionally hexane-1,6-diamine free base.
59. The process of any one of embodiments 50 to 58, wherein 0.6≤c_{L1}≤2.0, preferably 0.7≤c_{L1}≤1.6.
60. The process of any one of embodiments 50 to 59, wherein from 90 to 100 weight-%, preferably from 95 to 100 weight-%, more preferably from 99 to 100 weight-% of the liquid aqueous stream L₁ provided according to (i) consist of water, the at least carbon dioxide derivative of hexane-1,6-diamine and optionally hexane-1,6-diamine free base;
   wherein the liquid aqueous stream L₁ provided according to (i) has, at a temperature of L₁ of 25° C., a pH preferably in the range of from 6 to 10, more preferably in the range of from 7 to 9.5, more preferably in the range of from 7.5 to 9.
61. The process of any one of embodiments 50 to 60, wherein 10° C.≤T_{L1}<T, preferably 15° C.≤T_{L1}<T, more preferably 20° C.≤T_{L1}<T;
   wherein the temperature T according to (ii) is preferably in the range of from 90 to 190° C., more preferably in the range of from 100 to 180° C., more preferably in the range of from 110 to 170° C.
62. The process any one of embodiments 50 to 61, wherein c_{L2}≤0.08, preferably c_{L2}≤0.06, more preferably c_{L2}≤0.04, more preferably c_{L2}≤0.02.
63. The process of any one of embodiments 50 to 62, wherein the liquid aqueous stream L₁ is obtainable or obtained by a fermentation process and wherein the stream L₂ or a downstream stream obtained therefrom is subjected to extraction, wherein the downstream stream obtained from L₂ is the stream L₃ as defined in embodiment 50, the stream L₄ as defined in embodiment 51, or the stream L_{L3} as defined in embodiment 56.
64. A process for removing carbon dioxide from a liquid aqueous stream L₁ comprising at least one hexane-1,6-diamine species which comprises at least one carbon dioxide derivative of hexane-1,6-diamine, the process comprising
   (i) providing the liquid aqueous stream L₁ exhibiting a carbon dioxide loading c_{L1}, wherein c_{L1} is defined as n_{L1}(CO₂)/n_{L1}(HMDA) with n_{L1}(CO₂) being the molar amount of carbon dioxide comprised in the at least one carbon dioxide derivative of hexane-1,6-diamine in L₁ and n_{L1}(HMDA) being the molar amount of the at least one hexane-1,6-diamine species in L₁, wherein 0.5≤c_{L1}≤2.5, wherein the liquid aqueous stream L₁ to be subjected to heating according to (ii) has a temperature T_{L1}<T according to (ii);
   (ii) heating the liquid aqueous stream L₁ provided according to (i) to a temperature T≥90° C. at a pressure p≥0.5 bar(abs), obtaining a vapor stream V comprising carbon dioxide, and obtaining a liquid aqueous stream L₂ exhibiting a carbon dioxide loading c_{L2}, wherein c_{L2} is defined as n_{L2}(CO₂)/n_{L2}(HMDA) with n_{L2}(CO₂) being the molar amount of carbon dioxide comprised in the at least one carbon dioxide derivative of hexane-1,6-diamine in L₂ and n₂(HMDA) being the molar amount of the at least one hexane-1,6-diamine species in L₂, wherein c_{L2}≤0.1,
   wherein step (ii) is carried out in an evaporation unit comprising n serially coupled evaporation apparatuses Eⱼ, j=1 . . . n with n≥2, wherein an evaporation apparatus Eⱼ comprises heating means E_{Hj}, wherein an evaporation apparatus Eⱼ₊₁ is arranged downstream of an evaporation apparatus Eⱼ, wherein providing the liquid aqueous stream L₁ according to (i) comprises feeding the liquid aqueous stream L₁ for evaporation into the evaporation apparatus E₁;
   wherein for j=1 . . . n, (ii) comprises
   heating the liquid aqueous stream having been fed into the evaporation apparatus Eⱼ to a temperature Tⱼ at a pressure pⱼ in said evaporation apparatus Eⱼ, obtaining a vapor stream Vⱼ, the vapor stream Vⱼ having a temperature T_{Vj} and comprising carbon dioxide, and removing said vapor stream Vⱼ from the evaporation apparatus Eⱼ, and obtaining a liquid aqueous stream L₁ⱼ, the liquid aqueous stream L₁, having a temperature T_{L1j} and exhibiting a carbon dioxide loading c_{L1j}, wherein c_{L1j} is defined as n_{L1j}(CO₂)/n_{L1j}(HMDA) with n_{L1j}(CO₂) being the molar amount of carbon dioxide comprised in the at least one carbon dioxide derivative of hexane-1,6-diamine in L₁, and n_{L1j}(HMDA) being the molar amount of the at least one hexane-1,6-diamine species in L₁ⱼ, and removing said liquid aqueous stream L₁ⱼ from the evaporation apparatus Eⱼ; and
   feeding the liquid aqueous stream L₁ⱼ removed from Eⱼ into the evaporation apparatus Eⱼ₊₁ for evaporation and passing the vapor stream V_{Lj} as a heat source through the heating means E_{Hj+1} of the evaporation apparatus Eⱼ₊₁, and removing a stream W_{Lj+1} from the heating means E_{Hj+1};
   and wherein for j=n, (ii) comprises
   heating the liquid aqueous stream having been fed into the evaporation apparatus Eₙ to a temperature Tₙ at a pressure pₙ in said evaporation apparatus Eₙ, obtaining a vapor stream Vₙ=V, the vapor stream V having a temperature T_{V} and comprising carbon dioxide, and removing said vapor stream V from the evaporation apparatus Eₙ; and obtaining a liquid aqueous stream L₁ₙ=L₂, the liquid aqueous stream L₂ having a temperature T_{L2} and exhibiting the carbon dioxide loading c_{L2}, and removing said liquid aqueous stream L₂ from the evaporation apparatus Eₙ;
   wherein one parameter pair (Tⱼ;pⱼ) is the parameter pair (T;p) as defined in embodiment 1 with a temperature T≥90° C. and a pressure p≥0.5 bar(abs); preferably wherein n=7; more preferably wherein n=6; more preferably wherein n=5, more preferably wherein n=4, more preferably wherein n=3 or n=2.
65. The process of embodiment 64, wherein the parameter pair (T₁;p₁) is the parameter pair (T;p) as defined in embodiment 1 with a temperature T≥90° C. and a pressure p≥0.5 bar(abs);
   wherein T is preferably in the range of from 90 to 190° C., more preferably in the range of from 110 to 190° C., more preferably in the range of from 130 to 190° C.
66. The process of embodiment 64 or 65, wherein for j=1 . . . n−1, pⱼ₊₁<pⱼ; c_{L1j+1}<c_{L1j} and Tⱼ₊₁<Tⱼ; and wherein for j=1 . . . n−1, ΔT_{Ej+1}=T_{Vj}−T_{L1j+1} is preferably in the range of from 5 to 10 K.
67. The process of any one of embodiments 64 to 66, wherein the vapor stream V further comprises water in addition to carbon dioxide, the process further comprising subjecting the vapor stream V to condensation in a condenser C₁, obtaining a liquid stream L_{V} comprising water, being depleted in carbon dioxide and having a temperature T_{L}V, and further obtaining a vapor stream V_{V} comprising carbon dioxide and being depleted in water.
68. The process of any one of embodiments 64 to 66, further comprising passing the vapor stream V, obtained according to (ii.2), through a heat exchanger H₁, obtaining an optionally partially condensed stream VL having a temperature T_{VL}<T_{V}, wherein the process further comprises passing the liquid aqueous stream L₁ having a temperature T_{L10} through the heat exchanger H₁ obtaining the liquid aqueous stream L₁ having the temperature T_{L1} with T_{L1}>T_{L10}.
69. The process of any one of embodiments 64 to 68, wherein from 80 to 100 mol-%, preferably from 90 to 100 mol-%, more preferably from 95 to 100 mol-% of the at least one carbon dioxide derivative of hexane-1,6-diamine consist of one or more of hexamethylene-1,6-carbamate, hexamethylene-1,6-dicarbamate, hexamethylene-1,6-carbamate zwitterion, hexamethylene-1,6-carbonate, hexamethylene-1,6-bicarbonate, and hexamethy-lene-1,6-bis-bicarbonate;
   wherein preferably from 90 to 100 mol-%, more preferably from 95 to 100 mol-%, more preferably from 98 to 100 mol-% of the at least one hexane-1,6-diamine species consist of the at least one carbon dioxide derivative of hexane-1,6-diamine and optionally hexane-1,6-diamine free base.
70. The process of any one of embodiments 64 to 69, wherein 0.6≤c_{L1}≤2.0, preferably 0.7≤c_{L1}≤1.6.
71. The process of any one of embodiments 64 to 69, wherein from 90 to 100 weight-%, preferably from 95 to 100 weight-%, more preferably from 99 to 100 weight-% of the liquid aqueous stream L₁ provided according to (i) consist of water, the at least carbon dioxide derivative of hexane-1,6-diamine and optionally hexane-1,6-diamine free base;
   wherein the liquid aqueous stream L₁ provided according to (i) has, at a temperature of L₁ of 25° C., a pH preferably in the range of from 6 to 10, more preferably in the range of from 7 to 9.5, more preferably in the range of from 7.5 to 9.
72. The process of any one of embodiments 64 to 71, wherein 10° C.≤T_{L1}<T, preferably 15° C.≤T_{L1}<T, more preferably 20° C.≤T_{L1}<T;
   wherein the temperature T according to (ii) is preferably in the range of from 90 to 190° C., more preferably in the range of from 100 to 180° C., more preferably in the range of from 110 to 170° C.
73. The process any one of embodiments 64 to 72, wherein c_{L2}≤0.08, preferably c_{L2}≤0.06, more preferably c_{L2}≤0.04, more preferably c_{L2}≤0.02.

In the context of the present invention, a term “X is one or more of A, B and C”, wherein X is a given feature and each of A, B and C stands for specific realization of said feature, is to be understood as disclosing that X is either A, or B, or C, or A and B, or A and C, or B and C, or A and B and C. In this regard, it is noted that the skilled person is capable of transfer to above abstract term to a concrete example, e.g. where X is a temperature and A, B and C are concrete temperatures such as 10° C., 20° C., and 30° C. In this regard, it is further noted that the skilled person is capable of extending the above term to fewer specific realizations of said feature, e.g. “X is one or more of A and B” disclosing that X is either A, or B, or A and B, or to more specific realizations of said feature, e.g. “X is one or more of A, B, C and D”, disclosing that X is either A, or B, or C, or D, or A and B, or A and C, or A and D, or B and C, or B and D, or C and D, or A and B and C, or A and B and D, or B and C and D, or A and B and C and D.

### DESCRIPTION OF THE FIGURES

FIG. 1 is a schematic representation of a process according to the invention

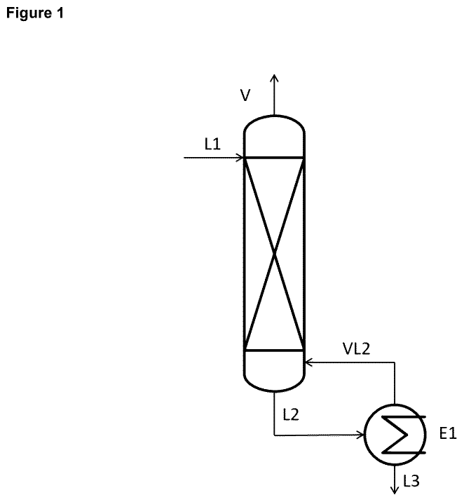

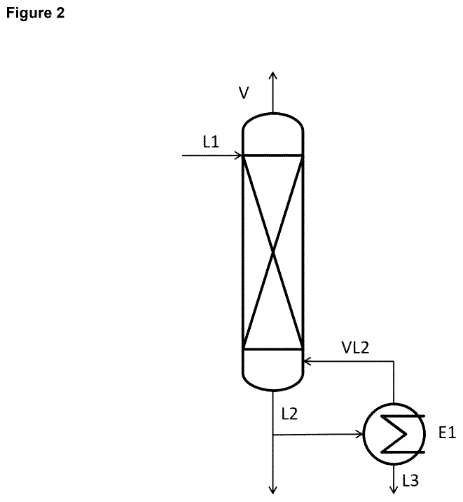

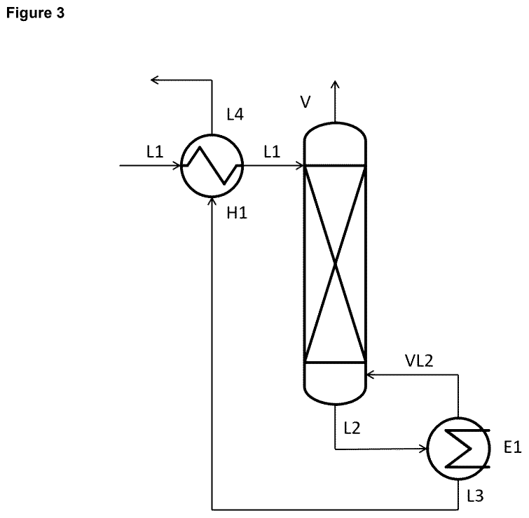

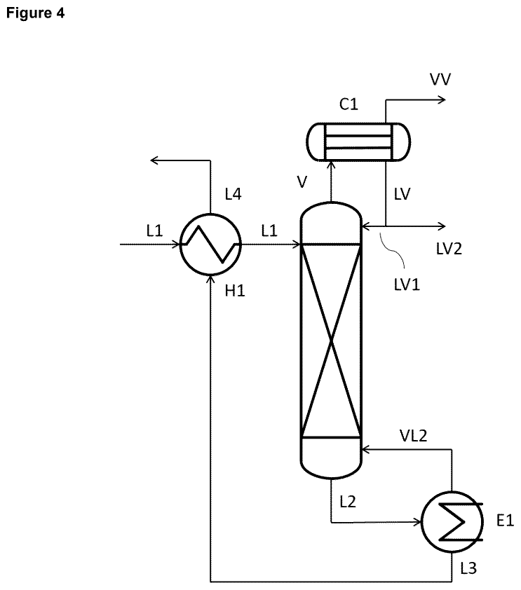

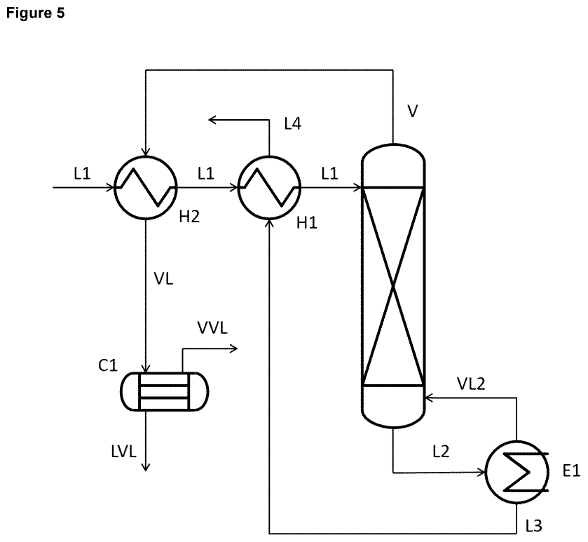

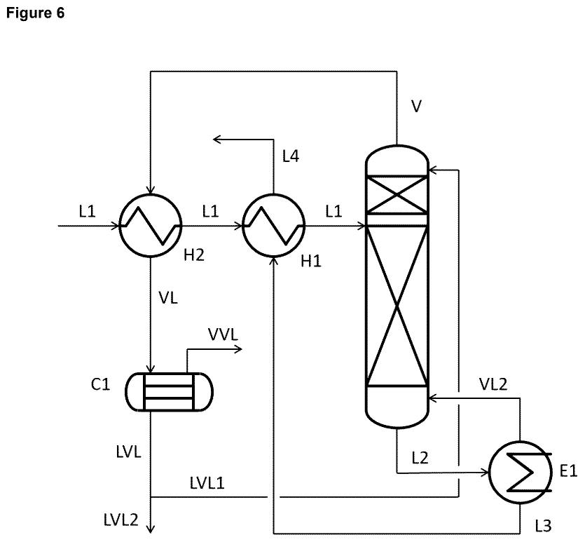

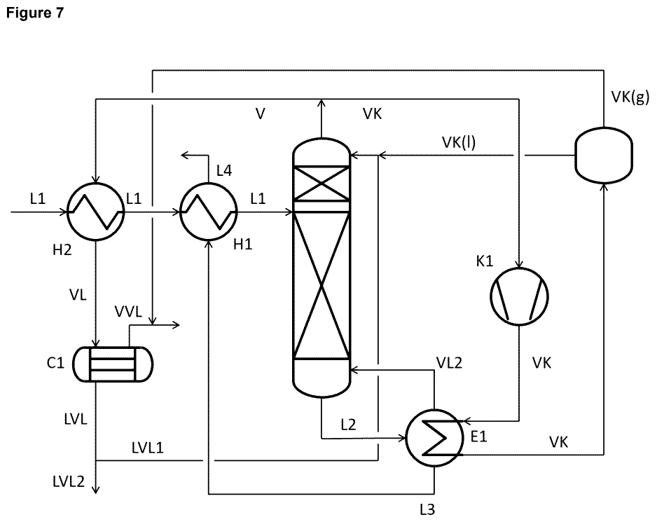

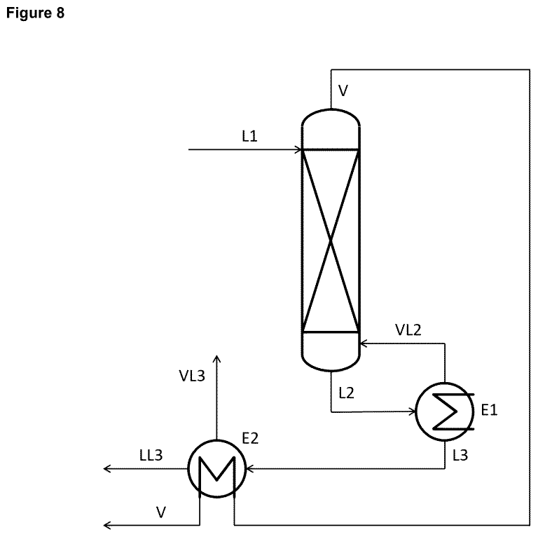

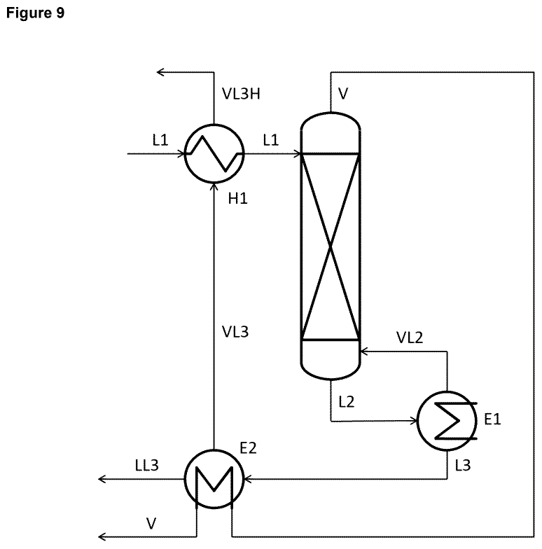

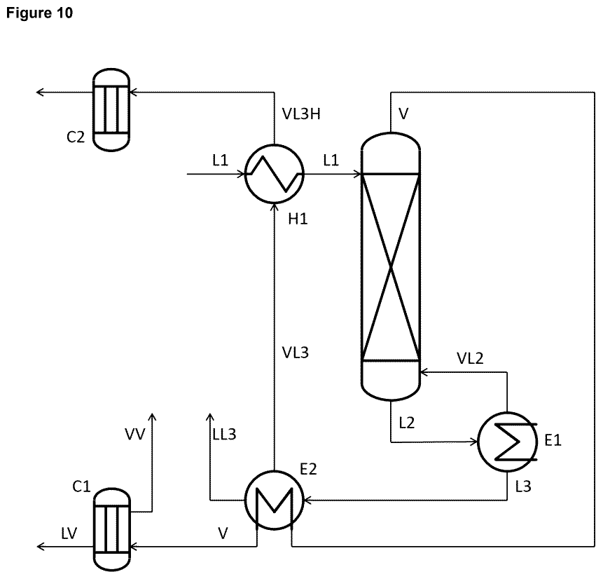

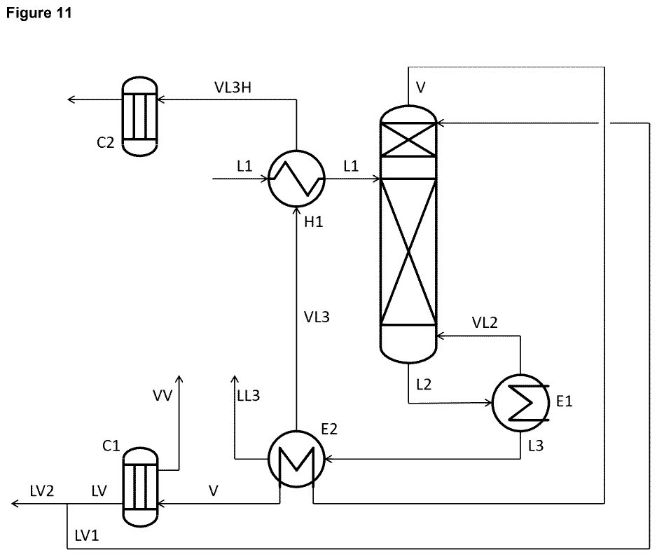

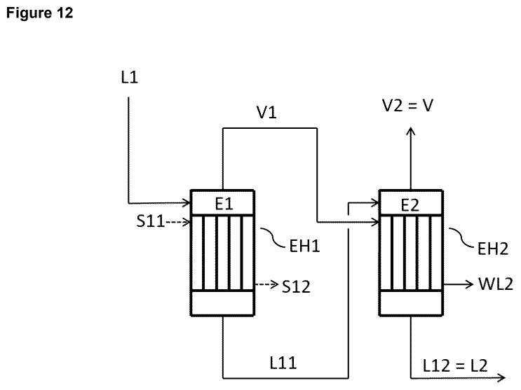

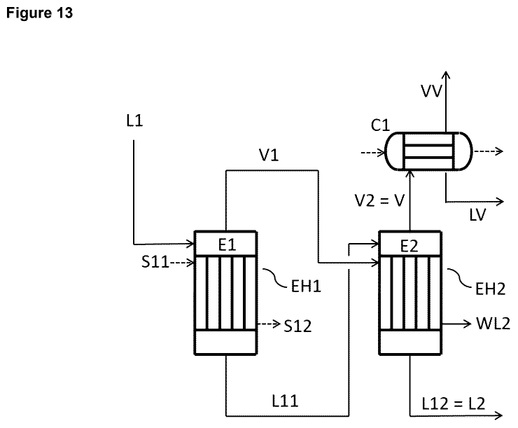

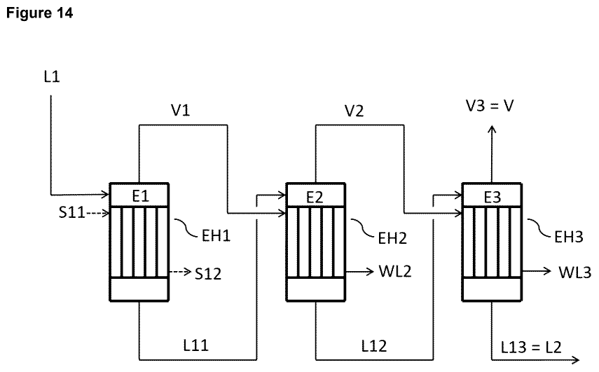

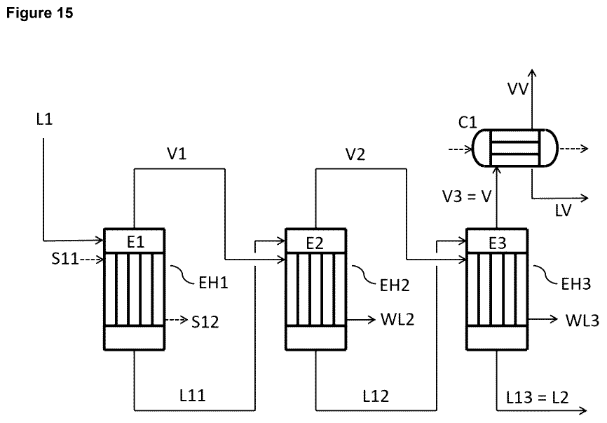

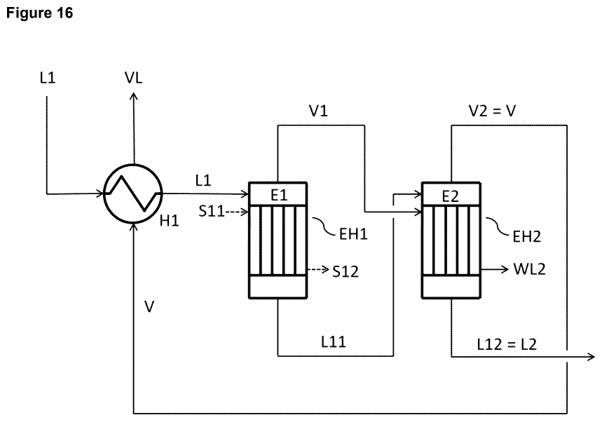

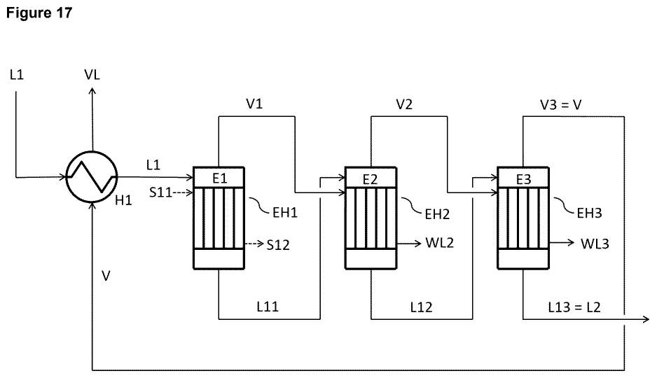

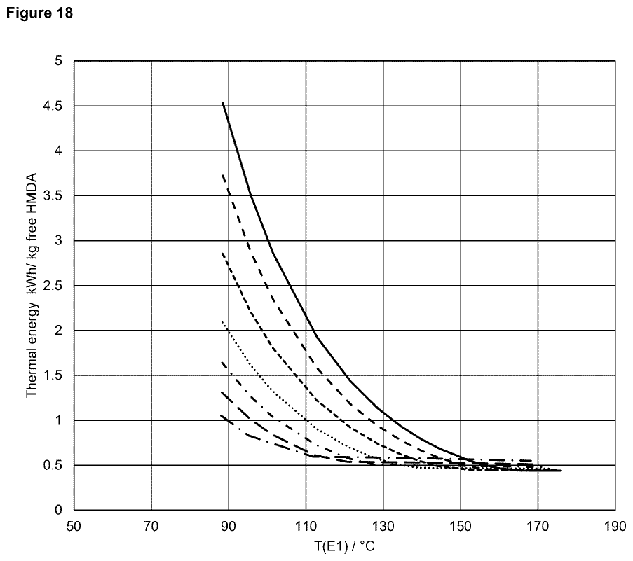

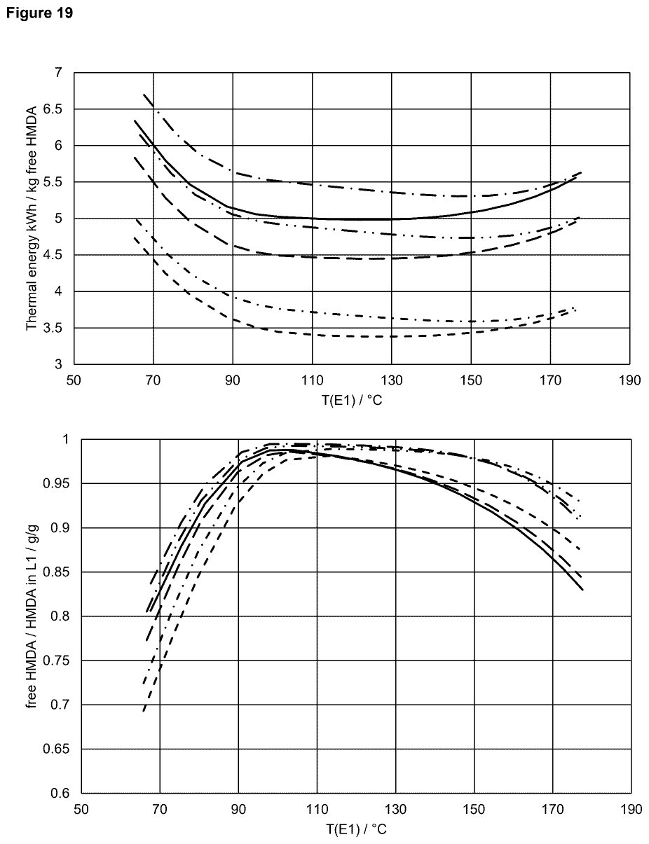

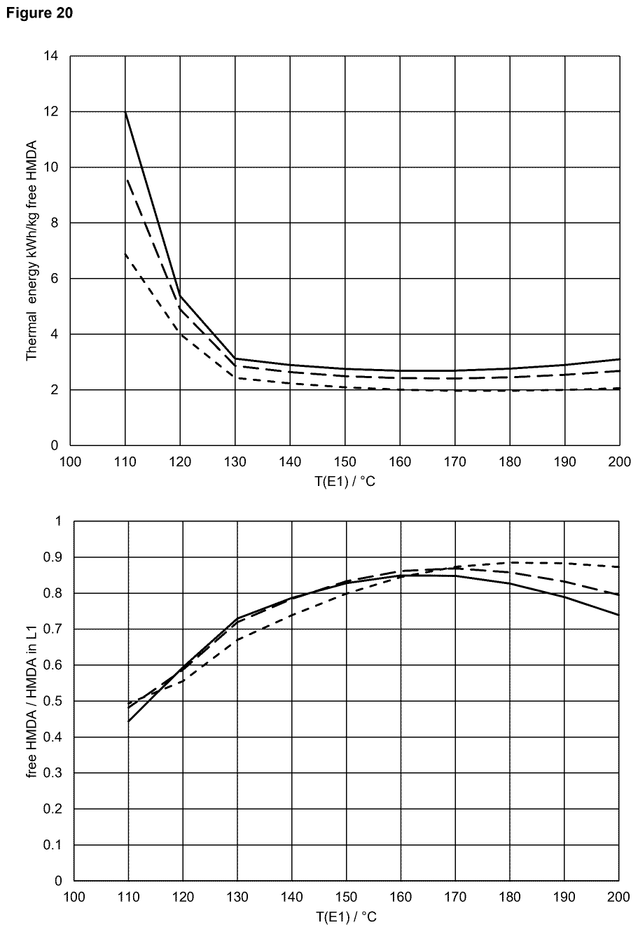

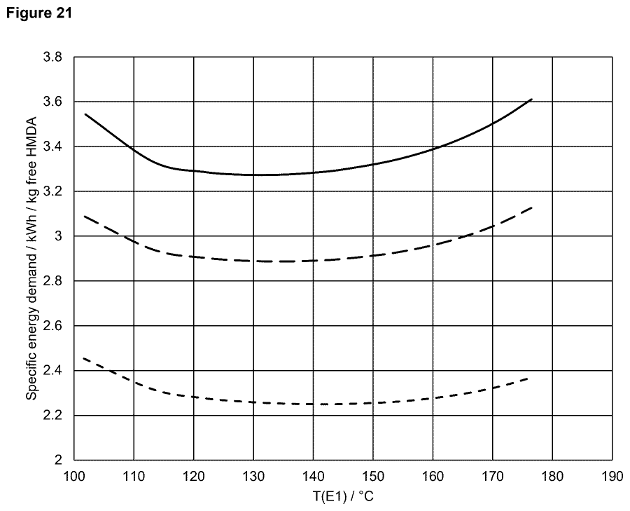

According to FIG. 1, an aqueous liquid stream L₁ comprising at least one hexane-1,6-diamine species which comprises at least one carbon dioxide derivative of hexane-1,6-diamine is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed and then passed to an evaporator E₁ in which L₂ is heated to obtain a vapor stream V_{L2} and a liquid stream L₃. The stream V_{L2} is then fed back to the bottoms section of the stripping column for heating purposes.

FIG. 2 is a schematic representation of a process according to the invention

According to FIG. 2, an aqueous liquid stream L₁ comprising at least one hexane-1,6-diamine species which comprises at least one carbon dioxide derivative of hexane-1,6-diamine is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed from the stripping column. The liquid stream L₂ is divided, and a portion of L₂ is passed to an evaporator E₁ in which said portion of L₂ is heated to obtain a vapor stream V_{L2} and a liquid stream L₃. The stream V_{L2} is then fed back to the bottoms section of the stripping column for heating purposes.

FIG. 3 is a schematic representation of a process according to the invention, including a preheating of the liquid stream L₁

According to FIG. 3, an aqueous liquid stream L₁ comprising at least one hexane-1,6-diamine species which comprises at least one carbon dioxide derivative of hexane-1,6-diamine is passed through a heat exchanger H₁, and a respectively heated stream L₁ is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed from the stripping column. The liquid stream L₂ is passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ is used as a heating medium in the heat exchanger H₁ for preheating the liquid stream L₁, and a respectively cooled stream L₄ is obtained.

FIG. 4 is a schematic representation of a process according to the invention, including a preheating of the liquid stream L₁ and further including reflux

According to FIG. 4, an aqueous liquid stream L₁ comprising at least one hexane-1,6-diamine species which comprises at least one carbon dioxide derivative of hexane-1,6-diamine is passed through a heat exchanger H₁, and a respectively heated stream L₁ is passed into the top section of a stripping column. At the top of the stripping column, a vapor stream V comprising carbon dioxide is obtained and removed. The vapour stream V is then passed to a condenser C₁, and from this condenser, a vapor stream V_{V} and a liquid stream L_{V} are obtained and removed from C₁. The liquid stream L_{V} is then divided into two liquid streams L_{V1} and L_{V2}, wherein the stream L_{V1} is passed back to the top section of the stripping column. At the bottoms of the stripping column, a liquid aqueous stream L₂, being depleted in carbon dioxide, is obtained and removed from the stripping column. The liquid stream L₂ is passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ is used as a heating medium in the heat exchanger H₁ for preheating the liquid stream L₁, and a respectively cooled stream L₄ is obtained.

FIG. 5 is a schematic representation of a process according to the invention, including a two-step preheating of the liquid stream L₁

According to FIG. 5, an aqueous liquid stream L₁ comprising at least one hexane-1,6-diamine species which comprises at least one carbon dioxide derivative of hexane-1,6-diamine is passed through a first heat exchanger H₂, and a respectively heated stream L₁ is passed through a second heat exchanger H₁, and a respectively heated stream L₁ is passed into the top section of a stripping column. At the top of the stripping column, a vapor stream V comprising carbon dioxide is obtained and removed. The vapor stream V is used as a heating medium in the first heat exchanger H₂ for the first preheating of the liquid stream L₁, and a partially condensed stream V_{L} is obtained which is then passed to a condenser C₁, and from this condenser, a vapor stream V_{VL} and a liquid stream L_{VL} are obtained and removed from C₁. At the bottoms of the stripping column, a liquid aqueous stream L₂, being depleted in carbon dioxide, is obtained and removed from the stripping column. The liquid stream L₂ is passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ is used as a heating medium in the second heat exchanger H₁ for preheating the liquid stream L₁, and a respectively cooled stream L₄ is obtained.

FIG. 6 is a schematic representation of a process according to the invention, including a two-step preheating of the liquid stream L₁ and further including reflux

According to FIG. 6, an aqueous liquid stream L₁ comprising at least one hexane-1,6-diamine species which comprises at least one carbon dioxide derivative of hexane-1,6-diamine is passed through a first heat exchanger H₂, and a respectively heated stream L₁ is passed through a second heat exchanger H₁, and a respectively heated stream L₁ is passed into the top section of a stripping column. In FIG. 6, the stripping column comprises an additional backwash section in the top section of the column, and the liquid stream L₁ is introduced into the column between the stripping section and said backwash section. At the top of the stripping column, a vapor stream V comprising carbon dioxide is obtained and removed. The vapor stream V is used as a heating medium in the first heat exchanger H₂ for the first preheating of the liquid stream L₁, and a partially condensed stream V_{L} is obtained which is then passed to a condenser C₁, and from this condenser, a vapor stream V_{VL} and a liquid stream LVL are obtained and removed from C₁. This liquid stream L₁ is then divided into two stream L_{VL1} and L_{VL2}, and the stream L_{VL1} is passed back to the top section of the stripping column. At the bottoms of the stripping column, a liquid aqueous stream L₂, being depleted in carbon dioxide, is obtained and removed from the stripping column. The liquid stream L₂ is passed to an evaporator E1 in which L₂ is heated to obtain a vapor stream V_{L2} and a liquid stream L₃. The stream V_{L2} is then fed back to the bottoms section of the stripping column for heating purposes. The liquid stream L₃ is used as a heating medium in the second heat exchanger H₁ for preheating the liquid stream L₁, and a respectively cooled stream L₄ is obtained.

FIG. 7 is a schematic representation of a process according to the invention, including a two-step preheating of the liquid stream L₁, further including reflux and top vapor compression

According to FIG. 7, an aqueous liquid stream L₁ comprising at least one hexane-1,6-diamine species which comprises at least one carbon dioxide derivative of hexane-1,6-diamine is passed through a first heat exchanger H₂, and a respectively heated stream L₁ is passed through a second heat exchanger H₁, and a respectively heated stream L₁ is passed into the top section of a stripping column. In FIG. 6, the stripping column comprises an additional backwash section in the top section of the column, and the liquid stream L₁ is introduced into the column between the stripping section and said backwash section. At the top of the stripping column, a vapor stream V comprising carbon dioxide is obtained and removed. From said top vapor stream V, a portion V_{K} is divided off while the remaining stream V is used as a heating medium in the first heat exchanger H₂ for the first preheating of the liquid stream L₁, and a partially condensed stream V_{L} is obtained which is then passed to a condenser C₁, and from this condenser, a vapor stream V_{VL} and a liquid stream LVL are obtained and removed from C₁. This liquid stream L₁ is then divided into two stream L_{VL1} and L_{VL2}, and the stream L_{VL}1 is passed back to the top section of the stripping column. At the bottoms of the stripping column, a liquid aqueous stream L₂, being depleted in carbon dioxide, is obtained and removed from the stripping column. The liquid stream L₂ is passed to an evaporator E₁ in which L₂ is heated to obtain a vapor stream V_{L2} and a liquid stream L₃. The stream V_{K} which was divided off the vapor stream V is passed through a compressor K₁, and a compressed stream V_{K} is obtained and used as a heating medium to heat up the stream L₃ in the evaporator E₁. The respectively obtained cooled stream V_{K} is then passed to a drum from which a liquid stream V_{K}(l) is passed to the top section of the stripping column above the backwash section, and from which a gaseous stream V_{K}(g) is combined with the vapor stream V_{VL} obtained from the condenser C₁. The stream V_{L2} which is obtained from the evaporator E₁ is fed back to the bottoms section of the stripping column for heating purposes. The liquid stream L₃ is used as a heating medium in the second heat exchanger H₁ for preheating the liquid stream L₁, and a respectively cooled stream L₄ is obtained.

FIG. 8 is a schematic representation of a process according to the invention, further including evaporation of the stream L₃

According to FIG. 8, an aqueous liquid stream L₁ comprising at least one hexane-1,6-diamine species which comprises at least one carbon dioxide derivative of hexane-1,6-diamine is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed and then passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ obtained from the evaporator E₁ is passed through an evaporator E₂, and a vapor stream V_{L3} and a liquid stream L_{L3} are obtained, wherein L_{L3} is depleted in carbon dioxide and concentrated with respect to hexane-1,6-diamine. As a heating medium for the evaporator E₂, the vapor stream V obtained from the top of the stripping column is used.

FIG. 9 is a schematic representation of a process according to the invention, further including evaporation of the stream L₃ and preheating of th stream L₁

According to FIG. 9, an aqueous liquid stream L₁ comprising at least one hexane-1,6-diamine species which comprises at least one carbon dioxide derivative of hexane-1,6-diamine is passed through a heat exchanger H₁, and a respectively heated stream L₁ is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed and then passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ obtained from the evaporator E₁ is passed through an evaporator E₂, and a vapor stream V_{L3} and a liquid stream L_{L3} are obtained, wherein L_{L3} is depleted in carbon dioxide and concentrated with respect to hexane-1,6-diamine. The vapor stream V_{L3} is used as a heating medium in the heat exchanger H₁ for the preheating of the liquid stream L₁, and a respectively cooled stream V_{L3H} is obtained. As a heating medium for the evaporator E₂, the vapor stream V obtained from the top of the stripping column is used.

FIG. 10 is a schematic representation of a process according to the invention, further including evaporation of the stream L₃ and preheating of the stream L₁, further including the condensation of vapor streams

According to FIG. 10, an aqueous liquid stream L₁ comprising at least one hexane-1,6-diamine species which comprises at least one carbon dioxide derivative of hexane-1,6-diamine is passed through a heat exchanger H₁, and a respectively heated stream L₁ is passed into the top section of a stripping column. At the top of the stripping column, a vapor stream V comprising carbon dioxide is obtained and removed. At the bottoms of the stripping column, a liquid aqueous stream L₂, being depleted in carbon dioxide, is obtained and removed and then passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ obtained from the evaporator E₁ is passed through an evaporator E₂, and a vapor stream V_{L3} and a liquid stream L_{L3} are obtained, wherein L_{L3} is depleted in carbon dioxide and concentrated with respect to hexane-1,6-diamine. The vapor stream V_{L3} is used as a heating medium in the heat exchanger H₁ for the preheating of the liquid stream L₁, and a respectively cooled stream V_{L3H} is obtained. As a heating medium for the evaporator E₂, the vapor stream V obtained from the top of the stripping column is used. The cooled stream V obtained from the evaporator E₂ is then passed to a condenser C1 from which a liquid stream L_{V} and a vapor stream V_{V} are obtained. The cooled stream V_{L3H} obtained from the heat exchanger H₁ is then passed through a condenser C₂ to obtain a liquid aqueous stream as a condensate.

FIG. 11 is a schematic representation of a process according to the invention, further including evaporation of the stream L₃ and preheating of the stream L₁, further including the condensation of vapor streams, and further including reflux

According to FIG. 11, an aqueous liquid stream L₁ comprising at least one hexane-1,6-diamine species which comprises at least one carbon dioxide derivative of hexane-1,6-diamine is passed through a heat exchanger H₁, and a respectively heated stream L₁ is passed into the top section of a stripping column. In FIG. 11, the stripping column comprises an additional backwash section in the top section of the column, and the liquid stream L₁ is introduced into the column between the stripping section and said backwash section. At the top of the stripping column, a vapor stream V comprising carbon dioxide is obtained and removed. At the bottoms of the stripping column, a liquid aqueous stream L₂, being depleted in carbon dioxide, is obtained and removed and then passed to an evaporator E₁ in which L₂ is heated to obtain a vapor stream V_{L2} and a liquid stream L₃. The stream V_{L2} is then fed back to the bottoms section of the stripping column for heating purposes. The liquid stream L₃ obtained from the evaporator E₁ is passed through an evaporator E₂, and a vapor stream V_{L3} and a liquid stream L_{L3} are obtained, wherein L_{L3} is depleted in carbon dioxide and concentrated with respect to hexane-1,6-diamine. The vapor stream V_{L3} is used as a heating medium in the heat exchanger H₁ for the preheating of the liquid stream L₁, and a respectively cooled stream V_{L3H} is obtained. As a heating medium for the evaporator E₂, the vapor stream V obtained from the top of the stripping column is used. The cooled stream V obtained from the evaporator E₂ is then passed to a condenser C1 from which a liquid stream L_{V} and a vapor stream V_{V} are obtained. The cooled stream V_{L3H} obtained from the heat exchanger H₁ is then passed through a condenser C₂ to obtain a liquid aqueous stream as a condensate. The liquid stream LV which is obtained from the condenser C1 is then divided into two streams L_{V1} and L_{V2}, and the stream LV1 is passed back as reflux to the top section of the stripping column, above the backwash section of the stripping column.

FIG. 12 is a schematic representation of a process according to the invention, including a two-stage evaporation of the stream L₁

According to FIG. 12, an aqueous liquid stream L₁ comprising at least one hexane-1,6-diamine species which comprises at least one carbon dioxide derivative of hexane-1,6-diamine is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₁, a vapor stream V₂ (V) and a liquid stream L₁₁ (L₂) are obtained, the liquid stream L₂ being depleted in carbon dioxide and enriched in hexane-1,6-diamine. From the heating means E_{H2}, a respectively cooled stream W₂ is obtained.

FIG. 13 is a schematic representation of a process according to the invention, including a two-stage evaporation of the stream L₁, further including the condensation of the vapor stream obtained from the downstream evaporation stage

According to FIG. 13, an aqueous liquid stream L₁ comprising at least one hexane-1,6-diamine species which comprises at least one carbon dioxide derivative of hexane-1,6-diamine is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₁, a vapor stream V₂ (V) and a liquid stream L₁₂ (L₂) are obtained, the liquid stream L₂ being depleted in carbon dioxide and enriched in hexane-1,6-diamine. From the heating means E_{H2}, a respectively cooled stream W₂ is obtained. The vapor stream V₂ (V) obtained from the evaporation apparatus E₂ is then passed to a condenser C₁ from which a condensed liquid stream L_{V} and a vapor stream V_{V} are obtained.

FIG. 14 is a schematic representation of a process according to the invention, including a three-stage evaporation of the stream L₁

According to FIG. 14, an aqueous liquid stream L₁ comprising at least one hexane-1,6-diamine species which comprises at least one carbon dioxide derivative of hexane-1,6-diamine is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₂, a vapor stream V₂ and a liquid stream L₁₂ are obtained, the liquid stream L₁₂ being depleted in carbon dioxide and enriched in hexane-1,6-diamine. From the heating means E_{H2}, a respectively cooled stream W_{L2} is obtained. The liquid stream L₁₂ is then passed into a third evaporation apparatus E₃ which is equipped with heating means E_{H3}. Through the heating means E_{H3}, the vapor stream V₂ obtained from the second evaporation apparatus E₂ is passed as a heating medium for heating up the liquid stream L₁₂. From the evaporation apparatus E₃, a vapor stream V₃ (V) and a liquid stream L₁₃ (L₂) are obtained, the liquid stream L₂ being depleted in carbon dioxide and enriched in hexane-1,6-diamine. From the heating means E_{H3}, a respectively cooled stream W_{L3} is obtained.

FIG. 15 is a schematic representation of a process according to the invention, including a three-stage evaporation of the stream L₁, further including the condensation of the vapor stream obtained from the downstream evaporation stage

According to FIG. 14, an aqueous liquid stream L₁ comprising at least one hexane-1,6-diamine species which comprises at least one carbon dioxide derivative of hexane-1,6-diamine is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₂, a vapor stream V₂ and a liquid stream L₁₂ are obtained, the liquid stream L₁₂ being depleted in carbon dioxide and enriched in hexane-1,6-diamine. From the heating means E_{H2}, a respectively cooled stream W_{L2} is obtained. The liquid stream L₁₂ is then passed into a third evaporation apparatus E₃ which is equipped with heating means E_{H3}. Through the heating means E_{H3}, the vapor stream V₂ obtained from the second evaporation apparatus E₂ is passed as a heating medium for heating up the liquid stream L₁₂. From the evaporation apparatus E₃, a vapor stream V₃ (V) and a liquid stream L₁₃ (L₂) are obtained, the liquid stream L₂ being depleted in carbon dioxide and enriched in hexane-1,6-diamine. From the heating means E_{H3}, a respectively cooled stream W_{L3} is obtained.

The vapor stream V₃ (V) obtained from the evaporation apparatus E₃ is then passed to a condenser C₁ from which a condensed liquid stream L_{V} and a vapor stream V_{V} are obtained.

FIG. 16 is a schematic representation of a process according to the invention, including a two-stage evaporation of the stream L₁, further including preheating of the stream L₁

According to FIG. 16, an aqueous liquid stream L₁ comprising at least one hexane-1,6-diamine species which comprises at least one carbon dioxide derivative of hexane-1,6-diamine is passed through a heat exchanger H₁, and the respectively heated liquid stream L₁ is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₁, a vapor stream V₂ (V) and a liquid stream L₁₁ (L₂) are obtained, the liquid stream L₂ being depleted in carbon dioxide and enriched in hexane-1,6-diamine. From the heating means E_{H2}, a respectively cooled stream W_{L2} is obtained. The vapor stream V₂ (V) obtained from the evaporation apparatus E₂ is used as a heating medium in the heat exchanger H₁ for the preheating of the liquid stream L₁, and a respectively cooled stream V_{L} is obtained.

FIG. 17 is a schematic representation of a process according to the invention, including a three-stage evaporation of the stream L₁, further including preheating of the stream L₁

According to FIG. 17, an aqueous liquid stream L₁ comprising at least one hexane-1,6-diamine species which comprises at least one carbon dioxide derivative of hexane-1,6-diamine is passed through a heat exchanger H₁, and the respectively heated liquid stream L₁ is passed into a first evaporation apparatus E₁ which is equipped with heating means E_{H1}. Through the heating means E_{H1}, a heating medium stream S₁₁ is passed and heats up the stream L₁. From the evaporation apparatus E₁, a vapor stream V₁ and a liquid stream L₁₁ are obtained, the liquid stream L₁₁ being depleted in carbon dioxide and enriched in hexane-1,6-diamine. From the heating means E_{H1}, a respectively cooled stream S₁₂ is obtained. The liquid stream L₁₁ is then passed into a second evaporation apparatus E₂ which is equipped with heating means E_{H2}. Through the heating means E_{H2}, the vapor stream V₁ obtained from the first evaporation apparatus E₁ is passed as a heating medium for heating up the liquid stream L₁₁. From the evaporation apparatus E₂, a vapor stream V₂ and a liquid stream L₁₂ are obtained, the liquid stream L₁₂ being depleted in carbon dioxide and enriched in hexane-1,6-diamine. From the heating means E_{H2}, a respectively cooled stream W₂ is obtained. The liquid stream L₁₂ is then passed into a third evaporation apparatus E₃ which is equipped with heating means E_{H3}. Through the heating means E_{H3}, the vapor stream V₂ obtained from the second evaporation apparatus E₂ is passed as a heating medium for heating up the liquid stream L₁₂. From the evaporation apparatus E₃, a vapor stream V₃ (V) and a liquid stream L₁₃ (L₂) are obtained, the liquid stream L₂ being depleted in carbon dioxide and enriched in hexane-1,6-diamine. From the heating means E_{H3}, a respectively cooled stream W_{L3} is obtained. The vapor stream V₃ (V) obtained from the evaporation apparatus E₃ is used as a heating medium in the heat exchanger H₁ for the preheating of the liquid stream L₁, and a respectively cooled stream V_{L} is obtained.

FIG. 18 is a representation of the results obtained from Example 2.1.

In the graph of FIG. 18, the temperature of the evaporator E₁ is plotted on the x axis whereas on the y axis, the thermal energy (in kWh) per kg of free HMDA is plotted. The following curves, attributed to the following percentages of HMDA are shown:

| | |
|---|---|
| 97% free HMDA | [symbol] |
| 95% free HMD | [symbol] |
| 90% free HMDA | [symbol] |
| 80% free HMDA | [symbol] |
| 70% free HMDA | [symbol] |
| 60% free HMDA | [symbol] |
| 50% free HMDA | [symbol] |

FIG. 19 is a representation of the results obtained from Examples 2.2 and 2.3.

In the graph of FIG. 19, the temperature of the evaporator E₁ is plotted on the x axis whereas on the y axis, the thermal energy (in kWh) per kg of free HMDA is plotted (upper graph) and the amount of free HMDA obtained per kg HMDA species in L₁ (lowergraph). The following curves, attributed to the following percentages of HMDA are shown:

| | |
|---|---|
| 60% water without backwashing | [symbol] |
| 70% water without backwashing | [symbol] |
| 80% water without backwashing | [symbol] |
| 60% water with backwashing | [symbol] |
| 70% water with backwashing | [symbol] |
| 80% water with backwashing | [symbol] |

FIG. 20 is a representation of the results obtained from Example 2.4.

In the graph of FIG. 20, the temperature of the evaporator E₁ is plotted on the x axis whereas on the y axis, the thermal energy (in kWh) per kg of free HMDA is plotted (upper graph) and the amount of free HMDA obtained per kg HMDA species in L₁ (lowergraph). The following curves, attributed to the following percentages of HMDA are shown:

| | |
|---|---|
| 60% water | [symbol] |
| 70% water | [symbol] |
| 80% water | [symbol] |

FIG. 21 is a representation of the results obtained from Example 2.5.

In the graph of FIG. 21, the temperature of the evaporator E₁ is plotted on the x axis whereas on the y axis, and the specific energy demand (in kWh) per kg of free HMDA is plotted. The following curves, attributed to the following percentages of HMDA are shown:

| | |
|---|---|
| 60% water | [symbol] |
| 70% water | [symbol] |
| 80% water | [symbol] |

### EXAMPLES

### 1. Determination of the Thermal Stability of Aqueous HMDA Solutions

For two aqueous solutions, on the one hand a CO₂-free 20 weight-% HMDA solution, on the other hand a 20 weight-% HMDA solution loaded with CO₂, the thermal stability was determined via dynamic differential calorimetry (DSC). For the CO₂-free solution, an exothermic decomposition reaction was observed at a temperature of 465° C. with an energy release of greater than 70 J/g; the CO₂-loaded HMDA solution (20 weight-% HMDA with a CO₂ loading og 1.0 mol CO₂ per mol HMDA) was thermally less resistant, and already at a temperature of 200° C. (onset temperature), an exothermic reaction with an energy release of 50 J/g was observed. It could be excluded that this was due to the release of CO₂ since such release is an endothermic process. Therefore, it could be concluded that—starting at a temperature of 200° C. —a CO₂-loaded HMDA in an aqueous solution is thermally unstable.

### 2. Examples Based on Process Simulations

Prior to process simulation, a thermodynamic model was developed based on which the phase equilibrium (vapor-liquid equilibrium) of the system CO₂-HMDA-H₂O could be described. The following chemical reaction in the liquid phase were taken into account:
Autoprotolysis of water: H₂O ↔H⁺+OH⁻
Formation of bicarbonate: CO₂+OH⁻↔HCO₃⁻
Formation of carbonate: HCO₃⁻↔CO₃²⁻+H⁺
1. Protonation of HMDA: HMD+H⁺↔HMDH⁺
2. Protonation of HMDA: HMDH⁺+H⁺↔HMD(H⁺)₂
Formation of monocarbamate: CO₂+HMD↔HMDCOO⁻+H⁺
Formation of bicarbamate: CO₂+HMDCOO⁻↔HMD(COO⁻)₂+H⁺
Formation of zwitterion: HMDCOO⁻+H⁺↔HMD(H⁺)(COO⁻)

For all process simulations, it was assumed that all reactions proceed sufficiently fast during stripping and, thus, could be regarded as equilibrium reaction. The model is based on gas solubility measurements for temperatures from 30 to 60° C. taken from the literature (Mondal et al., Fluid Phase Equilibria, 402 (2015) pages 102-112), supplemented by gas solubility measurement carried out at up to 160° C. by the inventors of the present invention. Further, also vapor-liquid equilibria of the binary system H₂O-HMDA were taken into account in order to allow for describing the volatility of HMDA in addition to the dissolved amount of CO₂. In this context, literature data were used (Rousseau et al., AIChE Symp. Ser. (1989) 85 (271), pages 73-78). For describing non-idealities—activity coefficients v in the liquid phase, the G^{E}-Modell according to Pitzer as formulated by Edwards was used (Edwards et al., AIChE J. (1975) 21(29) pages 248-259), and for describing of the gas phase, a cubic equation of state according to Redlich-Kwong-Soave. On this basis, it was possible to describe the separation of CO₂ and the preparation of a CO₂-free aqueous HMDA solution making use of a process simulation.

For all process simulations described hereinunder, the following liquid aqueous feed stream L₁ was used: aqueous solution, 10 weight-% in HMDA on a CO₂-free basis, having a temperature of 30° C. which was adjusted to a pH of 8.5 using CO₂.

### 2.1 Separation of CO₂ Using a Stripping Column

According to this example 2.1, CO₂ contained in the feed stream L₁ was separated using a stripping column configured as shown in FIG. 4. The stripping gas which was used was a stripping vapor prepared in situ during stripping, i.e. at the bottom section of the stripping column, a part of the aqueous HDA solution was evaporated. Due to the high boiling point of HMDA, essentially only water was evaporated. At the top of the stripping column, the pre-heated stream L₁ is passed into the column and passed through the column countercurrently to the stripping steam. The pre-heating of L₁ was achieved by heat integration according to which the hot HMDA solution depleted in CO₂ leaving E₁ as the stream L₃ transfers heat to the cold HMDA stream L₁ in H₁. At the top of the column, the vapor stream V was obtained which contains H₂O, CO₂ and traces of HMDA. This stream V was passed into the condenser C₁ where H₂O and HMDA were condensed out and recycled back into the column as stream L_{V}, and CO₂ leaves C₁ as gas (stream V_{V}). For simulation purposes, the condenser C₁ was operated at an exit temperature of 45° C.

As far as the stripping column is concerned, it was described in the model using a mass transfer-based model for which the height of the packings was 10 m with two-inch packings (one inch=2.54 cm). The diameter of the column was designed at 65% of the flood point of the column. The pressure at the top of the column was varied between 0.6 and 9 bar, corresponding to a temperature of the evaporator between 88 and 175° C. Relative to the amount of HMDA contained in L₁, the proportion of free HMDA was specified between 50 and 97%; thus, employing this stripping column, it was possible to significantly increase the yield in free HMDA. Further, it was found that with increasing temperature/increasing pressure in the stripping column, less energy was necessary to obtain free HMDA. Thus, e.g. for 97% free HMDA, the optimum temperature of the evaporator E₁ was found to be at least, preferably greater than 150° C. In this context, reference is made to FIG. 18 where the results of the simulation are shown.

### 2.2 Separation of CO₂ Using a Stripping Column

According to this example 2.2, CO₂ contained in the feed stream L₁ was separated using a stripping column configured as shown in FIG. 5. Compared to the stripping column as described in Example 2.1, the present configuration is a modified one according to which the goal of the process, namely increasing the HMDA content by evaporation and stripping off CO₂, can be achieved. The modification essentially concerns the heat integration concept involved and the fact that the condensate obtained from the condenser C₁ is not recycled back into the stripping column.

According to the present heat integration concept, the cold feed stream L₁ is first brought to a higher temperature using the vapor stream V comprising H₂O, CO₂ and traces of HMDA. The thus obtained heated stream L₁ is then, in a second heting step, brought to ist final temperature using the hot stream L₃ obtained from the evaporator E₁, which hot stream L₃ is depleted in CO₂ and contains the free HMDA. Once having entered the column, the stream L1, being passed though the column countercurrently to the stripping steam, is subjected to evaporation, resulting in the top stream V containing CO₂, H₂O and traces of HMDA.

As far as the stripping column is concerned, it was described in the model using a mass transfer-based model for which the height of the packings was 10 m with two-inch packings (one inch=2.54 cm). The diameter of the column was designed at 65% of the flood point of the column. The pressure at the top of the column was varied between 0.2 and 9 bar, corresponding to a temperature of the evaporator between 65 and 178° C. The degree of evaporation was specified via the water content of the concentrated HMDA solution depleted in CO₂ (stream L₂). The values of 80 weight-%, 70 weight-% and 60 weight. % warer content of L₂ were chosen. The respectively necessary energy input is higher than the energy input according to Example 2.4 (three stage evaporation); however, a higher yield in free HMDA was obtained. According to Example 2.4, a maximum of 88% free HMDA were obtained at a water content of 80 weight-%, whereas according to the present Example 2.2, 98% free HMDA at the water content of 80 weight-% were obtained. As shown in FIG. 19, with increasing pressure/temperature in the stripping column, the portion of evaporated HMDA increases and is removed with the condensate.

### 2.3 Separation of CO₂ Using a Stripping Column

According to this Example 2.3, the configuration according to Example 2.2 was enhanced by a backwash section at the top of the stripping column; reference is made to the respective FIG. 6. In particular, above the feeding point of the column where L₁ is introduced, a further packing bed is installed. In terms of the modelling of the column, a packing height of 3.5 m was chosen, as for the other packing section with 2 inch packings. The part stream L_{VL1} of the condensate stream LVL is passed into this backwash section. A reflux ratio of 1:10 was chosen.

According to this process configuration, it was possible to reduce the HMDA content in the condensate and, thus, the overall yield in free HMDA. As for the configuration according to Example 2.2 without backwash section, there is an optimum temperature range with regard to energy input and yield in free HMDA. Reference is made to FIG. 19 where the results are shown.

### 2.4 Separation of CO₂ Using Multistage Evaporation

According to this Example 2.4, the evaporation was carried out using a three-stage evaporation including heat integration, as shown in FIG. 17. According to this process configuration, only the evaporator E₁ needs to be heated with external energy (such as external steam (stream S₁₁), whereas the evaporators E₂ and E₃ are heated by the streams V₁ and V₂, respectively. For doing so, the temperature and the pressure, respectively, of the evaporator E₂ must be lower than those of E₁, and the temperature and the pressure, respectively, of the evaporator E₃ must be lower than those of E₂. Additionally, the feed stream L₁ is pre-heated in the heat exchanger H₁ using the stream V3=V. As driving temperature difference between condenser and evaporator, a value of 10 K was specified. The temperature of the evaporator E₁ was set, and the temperatures of the evaporators E₂ and E₃ were optimized so that the energy to be supplied externally in E₁ via S₁₁ becomes minimum. For the evaporator E₁, the temperature was varied from 110 to 200° C., wherein the water content of the concentrated HMDA solution depleted in CO2 was specified as 60 weight-%, 70 weight-% and 80 weight-%.

The results are shown in FIG. 20. The decisive parameter is the amount of HMDA which, after the separation of CO₂, is present as free HMDA. Other HMDA species such as protonated HMDA or the carbamate cannot be extracted in a subsequent extraction stage where only free HMDSA can be extracted into the organic phase. As shown in FIG. 20, for a value of 80 weight-% water, the energy input was 2 kWh per kg free HMDA; surprisingly, it was found that the yield in free HMDA was at 88%. (In this context, it is noted that for a respective one-stage evaporation, the energy input would be as high as 6.2 kWh per kg free HMDA at a yield of only 74%.

Generally, it was found that at higher temperatures and temperatures of at least 130° C., the specific energy demand of E₁ is minimized and at temperatures of at least 190° C., a slight increase of said energy demand is observed.

### 2.5 Separation of CO₂ Using Stripping and Evaporation

According to a further configuration of the process of the present invention, which is shown in FIG. 11, the advantages of stripping and evaporation were combined. In this process, the stripping column is essentially used to drive out CO₂, and in the downstream evaporation stage in E₂ which is operated at a reduced pressure compared to the upstream stripping column, excess steam obtained in V at the top of the stripping column is used for additionally evaporating water which is contained in L₃. According to the preferred embodiment, which is shown in FIG. 11, the respectively obtained stream V_{L3} is further used to pre-heat the feed stream L₁.

This process configuration thus combines the advantages of separating CO₂ in the stripping column and concentrating the bottoms stream obtained from said column with respect to its content of free HMDA. The results are shown in FIG. 21.

### LITERATURE CITED

US 2017/0369913 A1

1.-20. (canceled)

21. A process for removing carbon dioxide from a liquid aqueous stream L₁ comprising at least one hexane-1,6-diamine species which comprises at least one carbon dioxide derivative of hexane-1,6-diamine, the process comprising
(i) providing the liquid aqueous stream L₁ exhibiting a carbon dioxide loading c_{L1}, wherein c_{L1} is defined as n_{L1}(CO₂)/n_{L1}(HMDA) with n_{L1}(CO₂) being the molar amount of carbon dioxide comprised in the at least one carbon dioxide derivative of hexane-1,6-diamine in L₁ and n_{L1}(HMDA) being the molar amount of the at least one hexane-1,6-diamine species in L₁, wherein 0.5≤c_{L1}≤2.5, wherein the liquid aqueous stream L₁ to be subjected to heating according to (ii) has a temperature T_{L1}<T according to (ii); and
(ii) heating the liquid aqueous stream L₁ provided according to (i) to a temperature T>90° C. at a pressure p>0.5 bar(abs), obtaining a vapor stream V comprising carbon dioxide, and obtaining a liquid aqueous stream L2 exhibiting a carbon dioxide loading c_{L2}, wherein c_{L2} is defined as n_{L2}(CO₂)/n_{L2}(HMDA) with n_{L2}(CO₂) being the molar amount of carbon dioxide comprised in the at least one carbon dioxide derivative of hexane-1,6-diamine in L₂ and n_{L2}(HMDA) being the molar amount of the at least one hexane-1,6-diamine species in L₂, wherein c_{L2}≤0.1.

22. The process of claim 21, wherein from 80 to 100 mol-% of the at least one carbon dioxide derivative of hexane-1,6-diamine consist of one or more of hexamethylene-1,6-carbamate, hexamethylene-1,6-dicarbamate, hexamethylene-1,6-carbamate zwitterion, hexamethylene-1,6-carbonate, hexamethylene-1,6-bicarbonate, and hexamethy-lene-1,6-bis-bicarbonate.

23. The process of claim 21, wherein 0.6≤c_{L1}≤2.0.

24. The process of claim 21, wherein from 90 to 100 weight-% of the liquid aqueous stream L₁ provided according to (i) consist of water, the at least one carbon dioxide derivative of hexane-1,6-diamine and optionally hexane-1,6-diamine free base;
wherein the liquid aqueous stream L₁ provided according to (i) has, at a temperature of L₁ of 25° C., a pH in the range of from 6 to 10.

25. The process of claim 21, wherein 10° C.≤T_{L1}≤T, wherein the temperature T according to (ii) is in the range of from 90 to 190° C.

26. The process claim 21, wherein c_{L2}≤0.08.

27. The process of claim 21, wherein step (ii) is carried out in a stripping column using a stripping medium, wherein providing the liquid aqueous stream L₁ according to (i) comprises feeding the liquid aqueous stream L₁ into the stripping column, and wherein (ii) comprises
(ii.1) heating the liquid aqueous stream L₁ provided according to (i) in the stripping column to a temperature T of at least 90° C. at a pressure p of at least 0.5 bar(abs);
(ii.2) obtaining a vapor stream V at the top of the stripping column, the vapor stream V having a temperature T_{V} and comprising carbon dioxide, and removing said vapor stream V from the top of the stripping column; and
(ii.3) obtaining the liquid aqueous stream L₂ at the bottoms of the stripping column, the liquid aqueous stream having a temperature T_{L2} and comprising hexane-1,6-diamine free base, and removing said liquid aqueous stream L₂ from the bottoms of the stripping column;
the process further comprising
(iii.1) subjecting the liquid aqueous stream L2 obtained according to (ii.3) to evaporation in an evaporator E₁, obtaining an aqueous vapor stream V_{L2} having a temperature T_{VL2} and an aqueous liquid stream L3 having a temperature T_{L3}; and
(iii.2) feeding the aqueous vapor stream V_{L2} obtained according to (iii.1) back into the bottoms section of the stripping column.

28. The process of claim 27, further comprising
(iii.3) passing the aqueous liquid stream L3 obtained according to (iii.1) through a heat exchanger H₁, obtaining a stream L₄ having a temperature T_{L4} with T_{L4}<T_{L3}; and
(iii.4) passing the liquid aqueous stream L₁ having a temperature T_{L10}, prior to feeding it into the stripping column, through the heat exchanger H₁, obtaining the liquid aqueous stream L₁ having the temperature T_{L1} with T_{L1}>T_{L10};
wherein according to (iii.4), ΔT_{H1}=T_{L10}−T_{L4}.

29. The process of claim 27, wherein the vapor stream V obtained according to (ii.2) further comprises water in addition to carbon dioxide, the process further comprising
(iv.1) subjecting the vapor stream V obtained according to (ii.2) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

30. The process of claim 27, wherein the vapor stream V obtained according to (ii.2) further comprises water in addition to carbon dioxide, the process further comprising
(iv.0) passing the vapor stream V, obtained according to (ii.2), through a heat exchanger H₂, obtaining a partially condensed stream VL having a temperature T_{HVL}<T_{V}; and
(iv.1) subjecting the stream VL obtained according to (iv.0) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water;
wherein the process further comprises passing the liquid aqueous stream L₁ having a temperature T_{L100}, prior to passing it through the heat exchanger H₁ according to (iii.4), through the heat exchanger H₂ according to (iv.0), obtaining the liquid aqueous stream L₁ having the temperature T_{L10} with T_{L10}>T_{L100}.

31. The process of claim 30, further comprising, after (iv.0) and prior to (iv.1),
(iv.2) dividing the stream VL obtained according to (iv.0) in two streams VL₁ and VL₂, feeding the stream VL₁ back into the top section of the stripping column and subjecting the stream VL₂ as stream VL according to (iv.1) to condensation in the condenser C₁ according to (iv.2).

32. The process of claim 27, further comprising
(v.1) removing a vapor stream V_{K} having a temperature T_{VK} and comprising carbon dioxide from the top of the stripping column;
(v.2) passing the stream V_{K} removed from the top of the stripping column according to (v.1) through a compressor K₁, obtaining a compressed stream V_{K} having a temperature T_{CVK} with T_{CVK}>T_{VK}; and
(v.3) passing the compressed stream V_{K} as a heating medium through the evaporator E₁ according to (iii.1), obtaining a cooled compressed stream V_{K} comprising a liquid phase V_{K}(l) and optionally a vapor phase V_{K}(g).

33. The process of claim 27, further comprising
(iii.3) passing the vapor stream V, obtained according to (ii.2), through an evaporator E₂, obtaining the stream V having a temperature T_{EV}<T_{V}; and
(iii.4) subjecting the aqueous liquid stream L3 obtained according to (iii.1) to evaporation in the evaporator E₂ according to (iii.3), obtaining an aqueous vapor stream V_{L3} having a temperature T_{VL3} and an aqueous liquid stream L_{L3} having a temperature T_{LL3}.

34. The process of claim 33, wherein the vapor stream V obtained according to (iii.3) further comprises water in addition to carbon dioxide, the process further comprising
(iv.1) subjecting the vapor stream V obtained according to (iii.3) to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

35. The process of claim 21, wherein step (ii) is carried out in an evaporation unit comprising n serially coupled evaporation apparatuses Eⱼ, j=1 . . . n with n≥2, wherein an evaporation apparatus Eⱼ comprises heating means E_{Hj}, wherein an evaporation apparatus Eⱼ₊₁ is arranged downstream of an evaporation apparatus Eⱼ, wherein providing the liquid aqueous stream L₁ according to (i) comprises feeding the liquid aqueous stream L₁ for evaporation into the evaporation apparatus E₁;
wherein for j=1 . . . n, (ii) comprises
heating the liquid aqueous stream having been fed into the evaporation apparatus Eⱼ to a temperature Tⱼ at a pressure pⱼ in said evaporation apparatus Eⱼ, obtaining a vapor stream Vⱼ, the vapor stream Vⱼ having a temperature T_{Vj} and comprising carbon dioxide, and removing said vapor stream Vⱼ from the evaporation apparatus Eⱼ, and obtaining a liquid aqueous stream L₁ⱼ, the liquid aqueous stream L₁ⱼ having a temperature T_{L1j} and exhibiting a carbon dioxide loading c_{L1j}, wherein c_{L1j} is defined as n_{L1j}(CO₂)/n_{L1j}(HMDA) with n_{L1j}(CO₂) being the molar amount of carbon dioxide comprised in the at least one carbon dioxide derivative of hexane-1,6-diamine in L₁ⱼ and n_{L1j}(HMDA) being the molar amount of the at least one hexane-1,6-diamine species in L₁ⱼ, and removing said liquid aqueous stream L₁ⱼ from the evaporation apparatus Eⱼ; and
feeding the liquid aqueous stream L₁ⱼ removed from Eⱼ into the evaporation apparatus Eⱼ₊₁ for evaporation and passing the vapor stream V_{Lj} as a heat source through the heating means E_{Hj+1} of the evaporation apparatus Eⱼ₊₁, and removing a stream W_{Lj+1} from the heating means E_{Hj+1};
and wherein for j=n, (ii) comprises
heating the liquid aqueous stream having been fed into the evaporation apparatus Eₙ to a temperature T_{Ln} at a pressure pₙ in said evaporation apparatus Eₙ, obtaining a vapor stream Vₙ=V, the vapor stream V having a temperature T_{V} and comprising carbon dioxide, and removing said vapor stream V from the evaporation apparatus Eₙ; and obtaining a liquid aqueous stream L₁ₙ=L₂, the liquid aqueous stream L₂ having a temperature T_{L2} and exhibiting the carbon dioxide loading c_{L2}, and removing said liquid aqueous stream L₂ from the evaporation apparatus Eₙ;
wherein one parameter pair (Tⱼ;pⱼ) is the parameter pair (T;p) as defined in claim 21 with a temperature T≥90° C. and a pressure p≥0.5 bar(abs).

36. The process of claim 35, wherein the parameter pair (T₁;p₁) is the parameter pair (T;p) with a temperature T≥90° C. and a pressure p≥0.5 bar(abs).

37. The process of claim 35, wherein for j=1 . . . n−1, pⱼ₊₁<pⱼ; c_{L1j+1}<c_{L1j} and Tⱼ₊₁<Tⱼ.

38. The process of claim 35, wherein the vapor stream V further comprises water in addition to carbon dioxide, the process further comprising subjecting the vapor stream V to condensation in a condenser C₁, obtaining a liquid stream L_{V} comprising water, being depleted in carbon dioxide and having a temperature T_{LV}, and further obtaining a vapor stream V_{V} comprising carbon dioxide and being depleted in water.

39. The process of claim 35, further comprising passing the vapor stream V, obtained according to (ii.2), through a heat exchanger H₁, obtaining an optionally partially condensed stream VL having a temperature T_{VL}<T_{V}, wherein the process further comprises passing the liquid aqueous stream L₁ having a temperature T_{L10} through the heat exchanger H₁ obtaining the liquid aqueous stream L₁ having the temperature T_{L1} with T_{L1}>T_{L10}.

40. The process of claim 21, wherein the liquid aqueous stream L₁ is obtained by a fermentation process and wherein the stream L₂ or a downstream stream obtained therefrom is subjected to extraction, wherein the downstream stream obtained from L₂ is a stream L₃, a stream L₄ or a stream L_{L3}, wherein the stream L₃ has a temperature T_{L3}; the stream L₄ has a temperature T_{L4} with T_{L4}<T_{L3}; and the stream L_{L3} has a temperature T_{LL3}.
